(19) United States
(12) Patent Application Publication
(10) Pub. No.: US 2026/0074052 A1
(43) Pub. Date: Mar. 12, 2026

(54) System and Method for Trait Tracking

(71) Applicant: Daniel McCarron, Clearwater, FL (US)

(72) Inventor: Daniel McCarron, Clearwater, FL (US)

(21) Appl. No.: 18/830,673

(22) Filed: Sep. 11, 2024

(51) Int. Cl. G16H 20/70; G06F 3/0482

(52) U.S. Cl. CPC G16H 20/70; G06F 3/0482

(57) ABSTRACT

A method for identifying traits that need improvement includes providing a set of traits, dividing the set of traits into primary subsets and ranking each. Now, at least one trait is removed from each of the primary subsets and a plurality of secondary subsets is created, each taken from each of the primary subsets. Each secondary subset is rank ordered and at least one trait is removed from each to create tertiary subsets. Each tertiary subset has traits taken from each of the secondary subsets. Next, each tertiary subset is rank ordered, then for each top-rated trait, two traits are selected from either the top-rated trait or a fine-tuning trait and for the remaining traits, one trait from either the remaining trait or the fine-tuning trait. The selected traits and/or the fine-tuning traits are saved in a final subset of the traits.

### BACKGROUND OF THE INVENTION

There are many instances in which a person wants to make changes in their life. Very often, we set out do this by attempting to change a single behavior or a single habit. At times, we resort to expecting circumstances or others to change. For some, we realize that we have certain patterns of behavior, i.e. habits, that prevent or hinder us from having good relationships, functioning well in a career, or achieving certain desired outcomes.

One thing that is common for all people is that each person has a predominant set of traits that they regularly exhibit. For example, one person might more often respond to various sets of circumstances with anger while another person might respond to the same sets of circumstances more often with dishonesty, avoidance, or opportunism. Behind the habits that generally dictate a person's reaction and response to circumstance are a predominant set of traits. Although certain reactions are important in triggering certain behavioral responses to certain stimuli, many people often have traits, to which they habitually respond, that prevent or deter the achievement of desired outcomes, i.e., their common reactions and accompanying habitual behavioral responses are driven by a, or many, negative trait(s). For example, in disagreements between two people in a familial relationship, one might regularly resort to anger to convince the other that they are right. This may be effective for them to achieve that very short-term goal of being right. But if they are desiring that the relationship develop or improve (which would very likely be to their own benefit as well as that of the other person), that pattern of behavior is completely ineffective. With repetition, reacting with anger may even become a predominant trait of that person, spilling over into other facets of their life, and to other relationships, be they familial, social, or professional. Each time the individual reacts to anger in a negative manner, they reinforce not only the behavior; the patterned behavioral response to their angry reactions becomes a predominant trait, i.e., the person pervasively reacts with anger. The trait is, in and of itself, also a habit.

Often, habits are understood to be an action that is performed over and over again. For example, buying unnecessary things online. However, the traits driving actions are also habits. For an impulsive person, acting impulsively is both a trait and a habit.

Once traits are recognized as habits and their improvement is prioritized, improving the trait incentivizes changing other /bits/ behaviors linked to those traits, and effectively improving those linked habits and behaviors is much more easily accomplished.

Recognizing the traits which most negatively impact us as individuals is often difficult for people. Some lack an introspective nature, in which case they only become aware of a negative trait when it is pointed out by others (which is commonly taken as insult or criticism), or after experiencing a negative consequence. Even in an optimal case, where a person recognizes and desires changing a negative trait, the recognition is fleeting because after recognizing one, or a few, negative trait(s), it is very difficult to continually associate the traits which most negatively impact them with certain behaviors. The person will repeatedly exhibit, often unconsciously, the trained response. For example, when an impulsive person that has a compulsive shopping issue is about to hit the checkout button at the online retailer, the word “Impulsive” likely does not occur to them before they make the purchase, and, therefore, the person misses an opportunity to change their behavior. Instead, by hitting the “buy” button, they reinforce not only the behavior of hitting the button and the habit of buying things unnecessarily, they also further reinforce the already predominate trait of impulsiveness.

The use of raw willpower to change our habits often fails because there is not a competing priority which is compelling enough to overcome the habit we are trying to change; having a passing thought or even resolution to change a habit is often not enough to effect the change. By focusing on traits, we give ourselves a competing priority with the habit or behavior we are trying to overcome, which is the improvement of the trait itself. Instead of deriving pleasure from the act of buying something, we instead derive pleasure from not being impulsive.

What is needed is a system/method that will help people recognize the traits which they want to improve, that will reinforce the association of traits with certain other habits and behaviors, and that will provide feedback regarding these recognitions on a daily basis.

### SUMMARY OF THE INVENTION

The trait tracking system helps the user to identify the traits which most pervasively drive habitual behaviors that result in negative outcomes (i.e. the traits that the user wants/needs to improve) in a short exercise that is not a huge time burden, generally 25 to 45 minutes. By using the system, a customized list of these traits will be generated. This list of traits is referred to as the “significant traits” for that user. This exercise is referred to as the “scoping exercise.”

Once the list of significant traits has been generated, the user begins the activity of tracking their traits on a regular basis (at least several times a week), which takes 10 to 15 minutes and is referred to as the “tracking exercise.” This method consists of user's rating the prevalence of their significant traits in their day. By continuously performing this activity, users will reinforce the association of their significant traits with their habits, behaviors, and actions, such that when they are about to perform an action they have associated with a trait, they will think of the trait. When they recall the trait, the user has an opportunity, which they would not have otherwise had, to change the behavior about to be performed. This is called “self-interruption.”

With further continuous completion of the tracking exercise on the set of significant traits, self-interruption becomes more and more prevalent before the user completes actions they have associated with those traits. For example, assume that a user often spends money on unnecessary things and identifies that being Impulsive is a significant trait for them. The user begins to associate the term Impulsive with the behavior of clicking the “Buy Now” button in their online shopping app. After performing the tracking exercise for a couple or a few weeks (potentially sooner), the user will experience self-interruption when the user is about to hit any “Buy Now” button at an online retailer; the word Impulsive will be recalled by the user, and having that recollection affords an opportunity for the user to change their behavior. What's more, the user will likely experience self-interruption any time the user is about to spend money. This example only initially identified the behavior of pressing the “Buy Now” button. Actually, the behaviors associated with the trait the user is monitoring are more than just pressing a button, it is pressing a button and spending money; so, spending money generally will likely trigger self-interruption.

Though the performance of the tracking exercise will directly result in self-interruption, it cannot directly result in the changing of the user's will, as the user is ultimately responsible for changing their course of action. However, by consistently performing the tracking exercise, the motivation to change behaviors is greatly increased. This results from the increasing awareness of the pervasiveness of one or several of their significant traits on all aspects of their life. Where a user may have considered impulsiveness in connection with only a shopping habit, they will also see how being impulsive impacts other areas of their life as well. Potentially, in their intimate relationships, breaking promises to co-workers and family members, how they drive, the use of substances such as drugs or alcohol, general avoidance of responsibilities, etc. Once the pervasiveness of one or a few of the significant traits is recognized in their fuller embodiment, the self-interruption simply becomes so annoying and such a source of dissatisfaction that changing behaviors becomes much easier.

As the user changes behaviors to avoid the traits that the user is trying to improve, the user receives positive reinforcement at multiple times: first, when they experience self-interruption; second, at the moment immediately after the user changes a course of action; third when the user completes the tracking exercise for that day; and fourth when the user reviews reports showing progress for the day, week, month, etc.

In the above scenario, by refocusing or a shifting of priorities due to the gratification received by not being impulsive outweighing the immediate gratification from an unnecessary online purchase, the user will feel better by not making the impulsive purchase. Said another way, avoiding the habit of being impulsive becomes a higher priority than the habit of unnecessarily and impulsively buying things. As the user continues with the tracking exercise, the user solidifies this reorientation of priorities.

The trait tracker system provides a tool for the user to identify their significant traits and to generate a customized list of those significant traits. The trait tracker method provides the user with a way to track those traits so as to give themselves ample reinforcement of the association of traits with actions, such that behavior modification becomes more desirable and more manageable with little commitment of time or money.

The trait tracker system is used to identify anywhere from 12 to 16 significant traits. It is anticipated that a specific trait tracker system will generate lists of fewer or greater significant traits. This range was selected as it allows users to understand how their traits reinforce one another, as well as still be able to complete the tracking exercise in a short period of time. For instance, the impulsive person may be vain as well. A different person may be impulsive and anxious. Though both are impulsive, these are very different cases: one person is likely buying things to appear a certain way, the other is likely buying things to escape an emotional state. This drastic difference results only from linking the word Impulsive with one other trait. The difference is even more notable when the trait is linked with entirely different sets of other traits. It is the combination of using the trait tracker system to identify many significant traits and the method of tracking that provides users with an optimized (more effective and more efficient) experience and results.

There are two phases of each embodiment of the trait tracking system. The first phase is configuration phase, and the second phase is the daily tracking phase. The configuration phase relies upon the trait tracker system to generate a list of traits. A series of user interface screens are presented that couch the traits in a special way to allow proper ranking, usually taking a short amount of time, depending upon the user, anywhere from 25 minutes to 45 minutes, but generally under an hour. The second phase, or daily tracking phase, includes a method for tracking, which consists of the user entering their performance data each day based upon the actions they performed, or neglected to perform, that day. The user self-rewards themselves as they track each individual trait in which an action was changed for the better. Even if no actions were changed for the better, the user's tracking further reinforces the connection of traits and actions so as to make self-interruption more likely, and more pronounced when it does occur.

As there are many possible traits (272 unique, non-archaic, traits across the four embodiments of trait track system are identified), it would be unwieldy and complex if a user was to try and keep track of every possible trait. Therefore, for each embodiment of the trait tracking system, a specific set of traits have been identified for that specific trait tracking system. For example, there are 120 traits a user could possibly identify as significant for personal improvement, the embodiment specific to overall personal improvement. Still, 120 traits are too many for a user to keep in mind on a daily basis, and some of the 120 traits may not be important areas for improvement for specific users. For example, the trait of stubborn might be important to improve for one user that believes or feels that if they were they to improve this trait, it would positively impact them, but for a user that does not feel that they are stubborn to any extent in which it negatively impacts them, this trait may not be an area of concern for improvement.

To narrow the list of traits to something reasonable, the user first ranks a smaller set of traits (for an embodiment specific to overall personal improvement, a set of thirty traits), referred to as the “Base set.” As thirty traits is still a large enough number as to be difficult to rank all together, (and potentially create a sense of confusion in the user), the base set is broken down into smaller lists consisting of no more than six and no less than three traits (for the embodiment specific to overall personal improvement, the thirty traits are broken down into six lists of five traits). It is possible to set up the trait tracker system with lists consisting of two to any number of traits (up to the number of traits in a system). The range of three to six was determined to be the optimal for the user experience, regarding not being overwhelming yet still preserve efficiency in completing the scoping exercise. The user ranks the traits in each list, identifying the traits in order of most important to least important, where importance is defined by which trait, if improved, would have the greatest positive impact. This set of lists constitutes the primary series of the base set.

Based on the user's rankings of traits in the primary series of the base set, the user is presented with a secondary series of lists of traits from the base set. The secondary series consists of a smaller number of lists of three to six traits. Some traits which were ranked lowest in the first series are discarded, and those remaining are re-sequenced (for the embodiment specific to overall personal improvement, the secondary series consists of three lists of six traits each as the twelve lowest ranked traits from the primary series were discarded—two from each smaller list). Resequencing consists of moving words between lists, such that all the traits with higher rankings are compared to each other to ensure that a lower ranked trait from one list is not actually more important than the highest ranked trait of another list. For example, in embodiments specific to overall personal improvement, the first list of the secondary series includes the highest ranked trait from the first list of the primary series, the second most highly rated trait from the second list of the primary series, the third most highly ranked from the third list of the primary series, the first ranked from the fourth, etc. The second list of the secondary set includes the second most highly rated from the first list of the primary series, the third most highly ranked from the second list of the primary series, the first ranked of the third list of the primary series, etc., and so on with the third list of the secondary series. In this way, each of the highly rated traits from the lists in the primary series will be ranked against the other highly rated traits from the other lists of the primary series. The user then ranks the lists of the secondary series.

After ranking the secondary series of list of the base words, the user is then presented with a tertiary series of the base set. More traits are discarded (again the lowest ranked from the prior series of lists) and the remaining traits are again re-sequenced (at this point in the embodiment specific to personal improvement, 12 traits would remain to be ranked, and the tertiary series consists of two lists of six items each). The resequencing of the tertiary series consists of placing the highest ranked together and the remainder (not including those that are discarded) in another list, such that the appropriate ranking of the traits for the user can be determined. Upon completing the rankings of traits in the lists of the tertiary series, the user has now identified the most impactful traits (for them) from the base set and ranked them (the embodiment specific to personal improvement, the user has now identified the twelve most impactful traits from the base set and ranked those twelve traits). It is anticipated that the ranking activity involves fewer or greater numbers of sets based on the number of traits being included in the base words and the number of items to be presented in each list.

After completing the series of lists for the base set of traits, the user is presented with a final series consisting of lists with four traits per list. Each of these lists contains one of the of the twelve base traits identified from the base set with the three fine-tuning traits associated to that base trait. Each of these sets of four is a fine-tuning list. The user is presented with the twelve fine-tuning traits based on their results from the tertiary series. This final series is referred to as a “fine-tuning set” and contains only one series of lists. Within any trait tracking system, each unique trait in the base set is associated with three fine-tuning words (so for embodiment specific to personal improvement, there are ninety fine-tuning words). The fine-tuning traits associated with a base trait were determined based on the likelihood of either: the base trait being driven by or resulting from another trait and/or there being another underlying or related trait that is more important to the user. For instance, in the embodiment specific to personal improvement, the traits Undisciplined from the base set is associated with Unfocused, Insubordinate, and Hurried as fine-tuning traits. It may be that of these four, “Undisciplined” is that trait that, if improved, would have the greatest impact on the user. However, this final list presents the user with non-synonymous, yet potentially related traits to the base word for several purposes. Firstly, to avoid the necessity of having users needing to consider every potential trait that could be selected within a particular embodiment (the embodiment specific to personal improvement, eighteen base traits were discarded from the base set, which means the user is not going to have to consider the fifty-four fine-tuning words associated with those base words). Secondly, base traits were purposely identified to be base traits due a determination of their being more approachable and likely more common than those identified as fine-tuning traits; the user sees the traits that might be considered more extreme (the fine-tuning traits) within the context of a base word that they have already selected. As such, they may be more inclined to select the more appropriate, albeit more unpleasant trait, if they determine it is truly driving, or is more important than, the base trait they selected. Thirdly, because for some number of the highest ranked traits from the base set, the user's final list of significant traits will consist of two traits from lists from the fine-tuning set. For instance, in the embodiment specific to personal improvement, if Undisciplined is in a user's top four ranked traits from the base set (of the twelve which remain after going through the tertiary series), then the top two traits identified in the list consisting of that base trait and its fine-tuning traits will appear in their list of significant traits. It is possible to construct systems with fewer (1 or 2), more (more than 3), or no (0) fine-tuning associations. Systems with greater than three associations were determined to be less effective due to the associations between the base trait and its fine-tuning associates being weaker than in a system with three associations. Systems with less than three associations made the base sets larger (assuming the same number of traits) such that users would have to consider more traits than in a system with three associations.

An output list of the user's significant traits is generated that includes multiple traits from each list in the final series of the most impactful traits (i.e., two from each list in their fine-tuning set) and a single trait for the lower ranked, yet still significant, traits (i.e., one from each fine-tuning set). For the embodiment specific to personal improvement, eight of the sixteen significant traits will come from four fine-tuning lists and the remaining eight will come from the other eight fine-tuning lists. The user now has their custom list of significant traits that they work with during their daily exercise and feedback sessions.

The traits which are included in a specific embodiment of a system for trait tracking may or may not be classified into groups. In the embodiment specific to personal improvement, the traits are not classified. In an embodiment specific to professional improvement, the traits are classified into three groups: Quality, Initiative and Presentation. In an embodiment specific to overall academic improvement, traits are classified into three groups: Foundations, Analysis, and Communication. Grouping is helpful in that it helps users to think through how overall aspects of their performance are being impacted by their traits.

The tracking phase consists of ranking how their behavior reflected or improved each significant trait. Each of their traits is rated on a scale of “1” to “7”, where a rating of “1”, “2”, or “3” represents having been overwhelmed by, significantly impacted by, or just impacted by the significant trait, a rating of “4” represents not having been impacted by the significant trait, and a rating of 5 to 7 represents acting in a manner which improves upon the trait, to some extent, to a greater extent, or to an even greater extent. For example, if a user is tracking “Impulsive” as it relates to their shopping habits, and they did not buy something that day, but it never even occurred to them to purchase anything, they would rate themselves a “4.” If they refrained from making a minor purchase, a “5.” If they overcame multiple instances of wanting to purchase something more expensive, they may rate themselves a “6” or a “7.” If they ended up succumbing to the trait and it was something minor, they might rate themselves a “3.” If they spent more than they should have or can afford, they might rate themselves a “2” or a “1.” The scale could include more or less than 7 ratings.

Though it is not necessary in the tracking of traits, in some embodiments a unique aim for each trait is included within the trait tracking system. Aims are meant to encourage users to consider alternate ways of dealing with a circumstance.

After performing the tracking exercise, the trait tracking system provides users with reports showing progress in summarized and detailed formats for various spans of time: daily, weekly, monthly, or since inception.

In other words, during configuration of the trait tracking system for a given user and trait tracking application (currently, there are multiple trait tracking applications for various goals such as personal goals, professional goals, academic goals, and tracking character defects for recovering addicts), sets of traits are presented and the user ranks each set of traits (e.g., lists of three to six traits at a time) in the order of which would benefit the user the most. Once the user has used the trait tracking system to generate their list of significant traits, having identified the fifteen or so traits that, if improved, will have the most beneficial impact on them, they begin to track their traits by spending ten to fifteen minutes a day tracking progress for each of the identified traits. Users themselves reinforce improved behaviors and habits when they prioritize improving their traits by their actions, and when they track their traits. The use of the trait tracking system provides users with their customized list of significant traits, and the method used to track those traits, helps the users to monitor their improvements on their significant traits by providing daily, weekly, monthly, and since inception reporting on their progress. These reports, the product resulting from using the TraitTrack method, further reinforce the reorientation of priorities towards further improvement.

Although this disclosure uses one specific trait tracking application as an example (trait tracking for personal improvement), several other trait tracking applications are disclosed and it is fully anticipated and included here within that other trait tracking applications be created, for example, trait tracking applications that have sets of traits that match specific companies, organizations, or regions.

Once the user has used the trait tracking system to generate their list of traits that, if changed, will impact them the most, the user will revisit their trait tracking application, hopefully on a daily basis, and using the method will consider and record their progress, which includes tracking the occurrence of self-interruption, i.e. whether the user self-interrupts and thinks about their behavior. For example, if the user is about to perform a task that needs to be done and decides to do something else instead (e.g., they are procrastinating), if the user at least recognizes that they are procrastinating, they are at least self-interrupting, even if they still procrastinate, and such is recorded. If they actually change their habit and go back to perform the task that they would have procrastinated, this is recorded as well. The user starts to see their improvements by way of the application and begins to feel more rewarded by accomplishing changes in their habits rather than any good feelings achieved from performing their prior routines.

In one embodiment, a method for identifying traits that need improvement and monitoring of such improvements is disclosed including providing a set of traits (possible traits), dividing the set of traits into a plurality of primary subsets and ranking each primary subset in the plurality of primary subsets (e.g., from the highest to lowest, meaning the trait that, if improved, would likely be the most significant). Now, at least one trait is removed from each of the plurality of primary subsets and a plurality of secondary subsets is created from the plurality of primary subsets. Each secondary subset has traits taken from each subset of the primary subsets. Now, each secondary subset in the plurality of secondary subsets is rank ordered and at least one trait is removed from each of the plurality of secondary subsets to create a plurality of tertiary subsets from the plurality of secondary subsets. Each tertiary subset has the traits taken from each subset of the secondary subsets. Next, each tertiary subset in the plurality of tertiary subsets is rank ordered, then for each top-rated trait in each of the plurality of tertiary subsets, two traits are selected from either the top-rated trait or a fine-tuning trait from a list of fine-tuning traits that is associated with each trait and for a remaining traits in each of the plurality of tertiary subsets, one trait from either the remaining trait or the fine-tuning trait from the list of the fine-tuning traits that is associated with each trait. The selected traits and/or the fine-tuning traits are saved in a final subset of the traits.

In another embodiment, a system for identifying traits that need improvement and for monitoring of such improvements is disclosed including a device having a processor, a persistent storage, a non-transient memory, an input device, and a display with a set of traits stored in the persistent storage. Computer instructions running on the processor from the non-transient memory divides the set of traits into a plurality of primary subsets, displays each of the plurality of primary subsets on the display and receives inputs from the input device that provides ranks for each trait in each of the plurality of primary subsets. Next, the computer instructions remove at least one trait from each of the plurality of primary subsets then creates a plurality of secondary subsets from the plurality of primary subsets. Each secondary subset has the traits taken from each of the plurality of primary subsets. Now, the computer instructions display each of the plurality of secondary subsets on the display and receives the inputs from the input device that provides ranks for each trait in the plurality of secondary subsets. Next, the computer instructions removes at least one trait from each of the plurality of secondary subsets and creates a plurality of tertiary subsets from the plurality of secondary subsets. Each tertiary subset has the traits taken from each of the plurality of secondary subsets. The computer instructions displays each of the plurality of tertiary subsets on the display and receive the inputs from the input device that provides ranks for each trait in the plurality of tertiary subsets. Now, for each top-rated trait in each of the plurality of tertiary subsets, the computer instructions displays the each top-rated trait, receives the inputs selecting two traits from either the each top-rated trait or a fine-tuning trait from a list of the fine-tuning traits that is associated with the each top-rated trait, and saves selected traits in a final subset then for each remaining trait in each of the plurality of tertiary subsets, the computer instructions displays the each remaining trait, receives the inputs selecting one trait from either the each remaining trait or a fine-tuning trait from the list of the fine-tuning traits that is associated with the each remaining trait, and saves the selected trait in the final subset.

In another embodiment, a computer program product is disclosed including computing instructions stored on the one or more non-transitory computer-readable data storage media and executable by one or more processors of a device to perform the following:

Providing a set of traits, dividing the set of traits into a plurality of primary subsets and ranking each primary subset in the plurality of primary subsets, Next, removing at least one trait from each of the plurality of primary subsets and creating a plurality of secondary subsets from the plurality of primary subsets of which each secondary subset has traits taken from each of the plurality of primary subsets. Now, rank ordering each secondary subset in the plurality of secondary subsets, removing at least one trait from each of the plurality of secondary subsets and creating a plurality of tertiary subsets from the plurality of secondary subsets, of which each tertiary subset has the traits taken from each of the plurality of secondary subsets. Next, rank ordering each tertiary subset in the plurality of tertiary subsets and for each top-rated trait in each of the plurality of tertiary subsets, selecting two traits from either the top-rated trait or a fine-tuning trait from a list of the fine-tuning traits that is associated with each trait and for remaining traits in each of the plurality of tertiary subset of traits, selecting one trait from either the remaining trait or the fine-tuning trait from the list of the fine-tuning traits that is associated with each trait. Finally, saving the selected traits and/or the fine-tuning traits in a final subset.

### BRIEF DESCRIPTION OF DRAWINGS

The invention can be best understood by those having ordinary skill in the art by reference to the following detailed description when considered in conjunction with the accompanying drawings in which:

FIG. 1 illustrates a data connection diagram of the system for trait tracking.

FIG. 2 illustrates a schematic view of a typical smartphone or user device.

FIG. 3 illustrates a schematic view of a typical computer system such as a server or personal computer.

FIG. 4 illustrates an example list of traits useful for personal self-improvement.

FIG. 5 illustrates an example of traits useful for academic self-improvement.

FIG. 6 illustrates an example of traits useful for professional self-improvement.

FIG. 7 illustrates an example of traits useful for someone recovering from an addiction.

FIG. 8 illustrates a smartphone user interface of the system for trait tracking.

FIGS. 9, 9A, and 10-14 illustrate exemplary smartphone user interfaces of the system for trait tracking for the first phase of ranking of traits.

FIGS. 15, 16, and 17 illustrate exemplary smartphone user interfaces of the system for trait tracking for the second phase of ranking of traits.

FIGS. 18 and 19 illustrate exemplary smartphone user interfaces of the system for trait tracking for the third phase of ranking of traits.

FIGS. 20 and 20A illustrates an exemplary smartphone user interface of the system for trait tracking for selection of traits in fine tuning sets.

FIGS. 21A-21O illustrate a table of traits and aims as used by a personal trait tracker as used in the example user interfaces of FIGS. 9, 9A, and 10-19.

FIG. 22 illustrates an exemplary smartphone user interface of the system for trait tracking for finalizing a user's list of traits.

FIG. 23 illustrates an exemplary smartphone user interface of the system for trait tracking for capturing a user's activities for each trait.

FIG. 24 illustrates an exemplary smartphone user interface of the system for trait tracking for daily reporting.

FIGS. 25, 25A, and 25B illustrate exemplary smartphone user interfaces of the system for trait tracking for reporting progress to the user.

FIG. 26 illustrates an exemplary smartphone user interface of the system for trait tracking for sharing progress with others.

FIG. 27 illustrates an exemplary program flow of the system for trait tracking for ranking traits by a user.

FIG. 28 illustrates an exemplary program flow of the system for trait tracking for daily reporting.

FIG. 29 illustrates an exemplary program flow of the system for trait tracking for sharing progress.

FIGS. 30A-30S illustrate a table of traits and aims as used by an academic trait tracker application.

FIGS. 31A-31O illustrate a table of traits and aims as used by a professional trait tracker application.

FIGS. 32A-32G illustrate a table of traits and aims as used by a recovery trait tracker application.

### DETAILED DESCRIPTION OF THE INVENTION

Reference will now be made in detail to the presently preferred embodiments of the invention, examples of which are illustrated in the accompanying drawings. Throughout the following detailed description, the same reference numerals refer to the same elements in all figures.

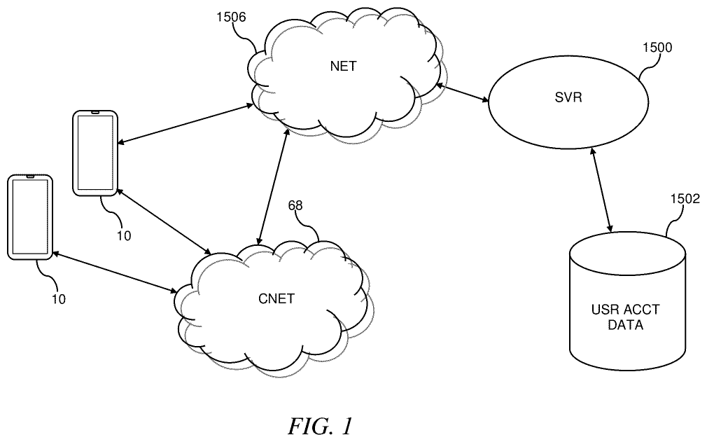

Referring to FIG. 1, a data connection diagram of the exemplary system for trait tracking is shown. In this example, one or more devices such as smartphones 10 communicate through the cellular network 68 and/or through a data network 1506 (e.g., the Internet) to a server computer 1500.

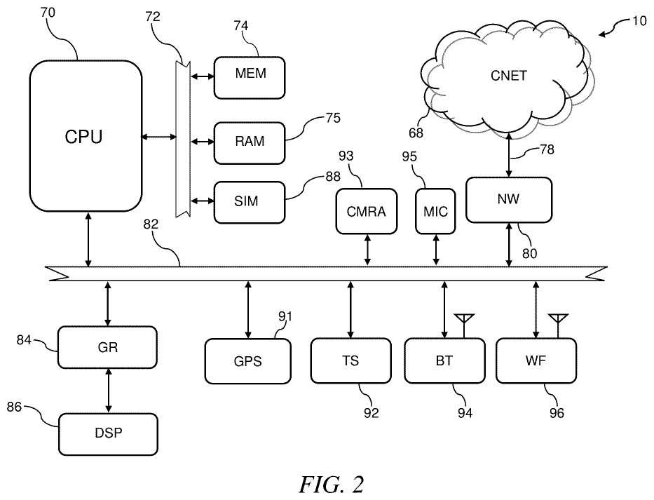

The server computer 1500 has access to user account data 1502 (e.g., from “cloud” storage or local storage) that is used to store user data and account information. Although a data path between the smartphones 10 and the server computer 1500 is shown going through the cellular network 68 and the data network 1506 as shown, any known data path is anticipated. For example, the Wi-Fi transceiver 96 (see FIG. 2) of the smartphone 10 is used to communicate directly with the wide area network 506, which includes the Internet, and, consequently, with the server computer 1500.

The server computer 1500 transacts with software running on the smartphones 10 through the network(s) 68/1506. The software (e.g., an application) presents menus to/on the smartphones 10, provides data to the smartphones 10, and communicates information to/from the server such as adding new user accounts or updating user account information.

Each user has an account having user account data 1502 stored and accessible by the server computer 1500 (e.g., username, credentials, preferences) as well as configuration data (e.g., the traits of which that user wants help), and daily data that is captured when the user works with their trait tracking application to record progress and displayed to show the user's daily, weekly, monthly, etc., progress. Billing information is also accessible by the server for billing the user, for example, for monthly use. It is also envisioned that groups of users will be consolidated into a single account, for example, a corporate account.

Further, it is also anticipated that upon completion of certain milestones or progress, the user will be rewarded with tangible (having value) or intangible (gold-star) rewards such as discounts on monthly services, retail shop cards (e.g., coffee), company logo trinkets, etc.

Referring to FIG. 2, a schematic view of a typical end-user device, a smartphone 10 is shown. Although any end-user device is anticipated (e.g., a personal computer or tablet computer), for clarity purposes, a smartphone 10 (and smartphone user interfaces) will be used in the remainder of the description.

The system for trait tracking is described using a processor-based end-user device (e.g., smartphone 10) for providing the login and user interfaces necessary for tracking trait activity, etc. The present invention is in no way limited to using a smartphone 10 and any similar device is anticipated (e.g., cellular phone, portable digital assistant, tablet computer, notebook computer, etc.).

The example smartphone 10 represents a typical device used for accessing user interfaces of the system for trait tracking. This smartphone 10 is shown as an example. Different architectures are known that accomplish similar results in a similar fashion and the present invention is not limited in any way to any device, smartphone 10 system architecture or implementation. In this exemplary smartphone 10, a processor 70 executes or runs programs in a random-access memory 75. The programs are generally stored within a persistent memory 74 and loaded into the random-access memory 75 when needed. Also accessible by the processor 70 is a SIM (subscriber information module) card 88 having a subscriber identification and often persistent storage. The processor 70 is any processor, typically a processor designed for phones. The persistent memory 74, random access memory 75, and SIM card are connected to the processor by, for example, a memory bus 72. The random-access memory 75 is any memory suitable for connection and operation with the selected processor 70, such as SRAM, DRAM, SDRAM, RDRAM, DDR, DDR-2, etc. The persistent memory 74 is any type, configuration, capacity of memory suitable for persistently storing data, for example, flash memory, read only memory, battery-backed memory, etc. In some exemplary smartphones 10, the persistent memory 74 is removable, in the form of a memory card of appropriate format such as SD (secure digital) cards, micro-SD cards, compact flash, etc.

Also connected to the processor 70 is a system bus 82 for connecting to peripheral subsystems such as a cellular network interface 80, a graphics adapter 84 and a touch screen interface 92. The graphics adapter 84 receives commands from the processor 70 and controls what is depicted on the display 86. The touch screen interface 92 provides navigation and selection features.

In general, some portions of the persistent memory 74 and/or the SIM card 88 is used to store programs, executable code, and data, etc. In some embodiments, other data is stored in the persistent memory 74 such as audio files, video files, text messages, etc.

The peripherals are examples and other devices are known in the industry such as Global Positioning Subsystem 91, speakers, microphones, USB interfaces, camera 93, microphone 95, Bluetooth transceiver 94, Wi-Fi transceiver 96, image sensors, temperature sensors, etc., the details of which are not shown for brevity and clarity reasons.

The cellular network interface 80 connects the smartphone 10 to the cellular network 68 through any cellular band and cellular protocol such as GSM, TDMA, LTE, 4G, 5G, etc., through a wireless medium 78. There is no limitation on the type of cellular connection used. The cellular network interface 80 provides voice call, data, and messaging services to the smartphone 10 through the cellular network 68.

For local communications, many smartphones 10 include a Bluetooth transceiver 94, a Wi-Fi transceiver 96, or both. Such features of smartphones 10 provide data communications between the smartphones 10 and data access points and/or other computers such as a personal computer (not shown).

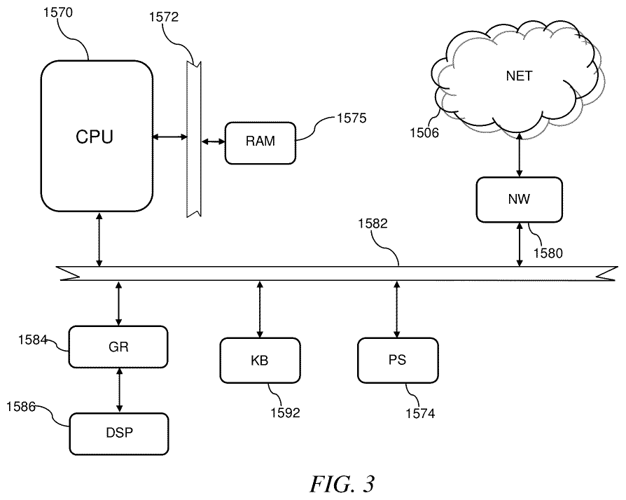

Referring to FIG. 3, a schematic view of a typical computer system (e.g., server computer 1500) is shown. The exemplary server computer 1500 represents a typical computer system used for back-end processing, generating reports, displaying data, etc. This exemplary computer system is shown in its simplest form. Different architectures are known that accomplish similar results in a similar fashion and the present invention is not limited in any way to any particular computer system architecture or implementation. In this exemplary computer system, a processor 1570 executes or runs programs in a random-access memory 1575. The programs are generally stored within a persistent memory 1574 and loaded into the random-access memory 1575 when needed. In some embodiments, the persistent memory 1574 contains the user account data while in other embodiments, the user account data 1502 is stored in any known storage such as cloud-based storage.

The processor 1570 is any processor, typically a processor designed for computer systems with any number of core processing elements, etc. The random-access memory 1575 is connected to the processor by, for example, a memory bus 1572. The random-access memory 1575 is any memory suitable for connection and operation with the processor 1570, such as SRAM, DRAM, SDRAM, RDRAM, DDR, DDR-2, etc. The persistent memory 1574 is any type, configuration, capacity of memory suitable for persistently storing data, for example, magnetic storage, flash memory, read only memory, battery-backed memory, magnetic memory, etc. The persistent memory 1574 (e.g., disk storage) is typically interfaced to the processor 1570 through a system bus 1582, or any other interface as known in the industry.

Also shown connected to the processor 1570 through the system bus 1582 is a network interface 1580 (e.g., for connecting to a data network 1506), a graphics adapter 1584 and a keyboard interface 1592 (e.g., Universal Serial Bus—USB). The graphics adapter 1584 receives commands from the processor 1570 and controls what is depicted on a display 1586. The keyboard interface 1592 provides navigation, data entry, and selection features.

In general, some portion of the persistent memory 1574 is used to store programs, executable code, data, and other data, etc.

The peripherals are examples and other devices are known in the industry such as pointing devices, touch-screen interfaces, speakers, microphones, USB interfaces, Bluetooth transceivers, Wi-Fi transceivers, image sensors, temperature sensors, etc., the details of which are not shown for brevity and clarity reasons.

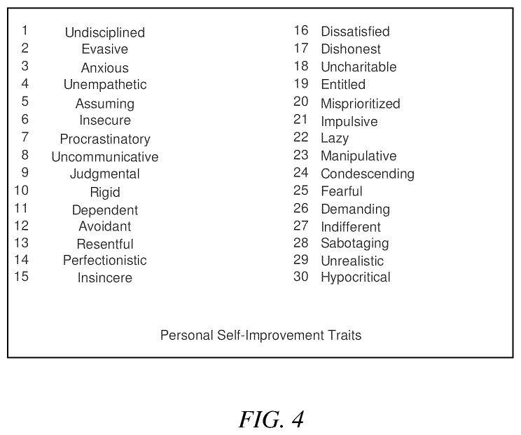

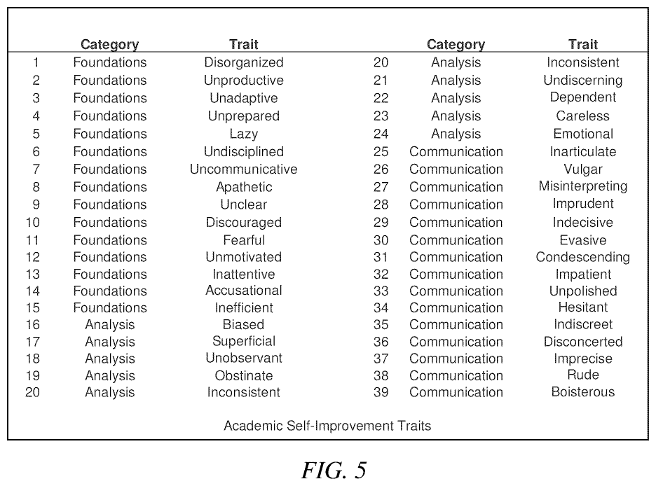

FIG. 4 illustrates one set of traits useful for general self-improvement, an application of which will be referred to as a personal trait tracker. This set of traits will be used throughout this description for explanation purposes, though FIGS. 5-7 have equally important sets of traits for other trait tracker applications and other sets of traits are fully anticipated. Different sets of traits are used depending upon the type of self-improvement that is desired. For example, for someone looking to improve themselves professionally, traits such as disorganized or unproductive are important, but not for someone who is trying to recover from an addiction. For a recovering addict, improving traits such as self-pitying and sabotaging are far more important than whether they are organized or productive.

It is important to note that there are countless numbers of traits possible and the sets of traits for any given implementation of the disclosed method and system are selected from the possible traits to be best suited to the particular application. For example, in selecting traits for a recovery version of the trait tracker application, traits such as self-pitying and envious are important as many who have addictions might exhibit such traits and might want to correct those traits, while traits such as disorganized and rude might not be traits that a recovering addict might be interested in working to improve.

It should be noted that the sets provided in this document are representative sets of current practice and are not meant to be exhaustive or absolute as it is envisioned that traits be added or removed from any of the sets that are shown. Further, it is fully envisioned that in some embodiments, other custom sets be created for specific targets. For example, a set of traits specifically for firefighters or police officers, or soldiers, or doctors, or nurses. Other examples are company-specific sets targeted to specific traits that need improvement customized to the particular company. For example, a delivery company might include traits related to driving skills, courtesy to clients, care in handling of packages, etc.

FIG. 5 illustrates one list of traits useful for academic self-improvement. In this list, there are 39 traits listed and subdivided into categories of “foundations,” “analysis,” and “communications.”

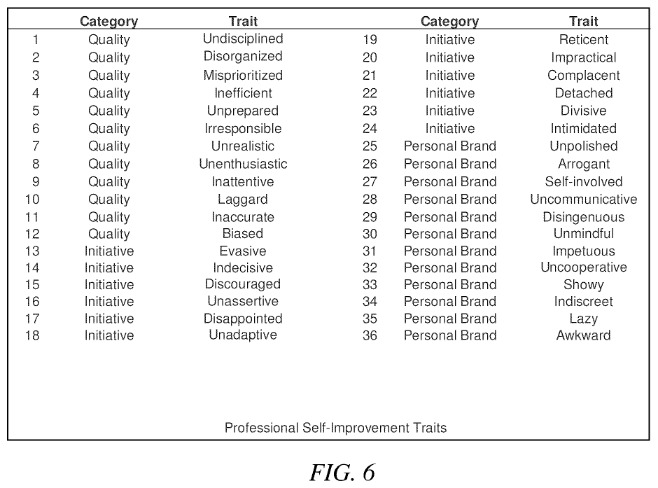

FIG. 6 illustrates one list of traits useful for professional self-improvement. In this list, there are 36 traits listed and subdivided into categories of “quality,” “initiative,” and “presentation.”

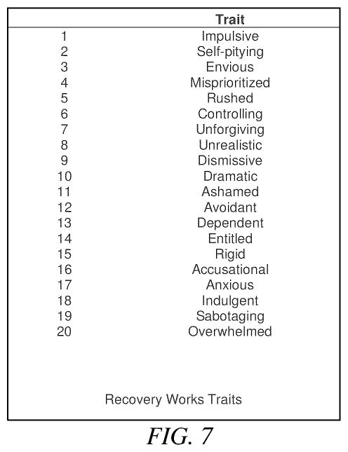

FIG. 7 illustrates one list of traits useful for someone recovering from an addiction. Note that this list is purposely short and concentrates on traits that a person who is recovering from any type of addiction might be concerned about.

Note that all user interfaces shown in subsequent figures are for explanation and clarity and many different user interfaces are anticipated and included here within. Note that various navigation features of the user interface such as navigation and exit features are purposely left out for clarity and brevity reasons.

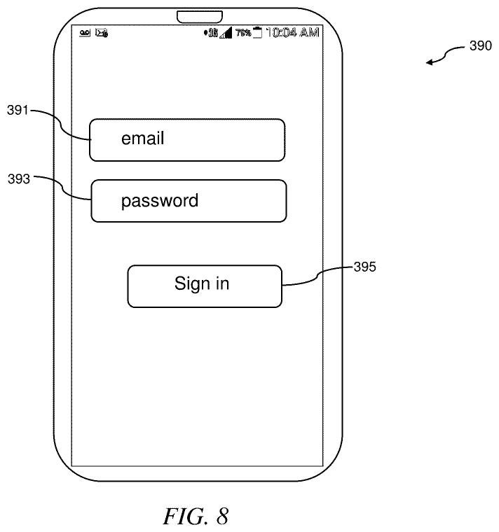

Referring to FIG. 8, a first user interface 390 provides a way for the user to login to the trait tracker system, providing, for example, their email address 391 (or username) and a password 393, then invoking the sign-in feature 395. This first user interface 390 is shown for completeness and is in no way limiting the trait tracker application to any specific login mechanism, etc.

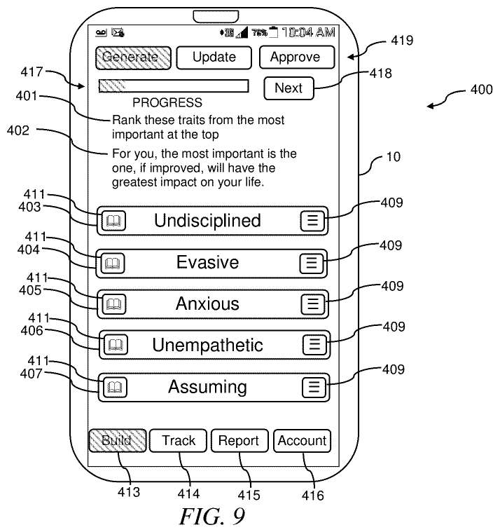

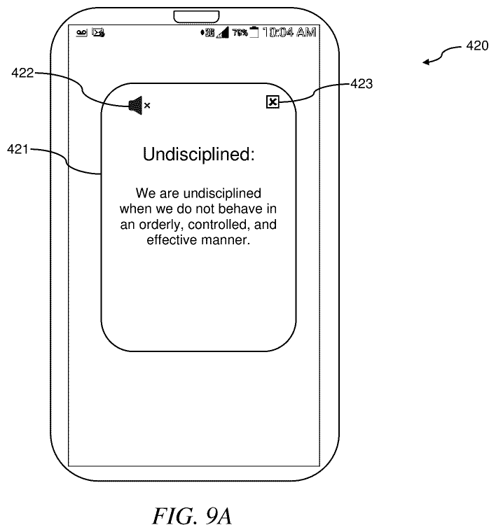

Referring to FIG. 9, a first primary user interface 400 of the system for trait tracking is shown. This first primary user interface 400 includes an action section 419 that informs the user as to what steps they are taking, in this example, the user is in the progress of generating their key traits (generating, approving, updating). Also shown is a progress indicator 417 showing the user how far along they are in selecting their traits (e.g., a first set of traits 403/404/405/406/407 in the first primary user interface 400) and a next directive 418 that the user invokes to select their next set of traits. In the example trait tracking application presented, there are six primary user interfaces 400/420/440/460/480/520 as shown in FIGS. 9-14.

Note that there are several potential invocations of the trait tracking application, for example, one for general self-improvement, one for professional self-improvement, one for a recovering addict, etc. For each, different sets of possible traits are anticipated as certain traits are specific to certain users. For example, for general self-improvement, there are 30 traits; for professional self-improvement, there are 36 traits, for academic self-improvement, there are 39 traits; and for recovery work, there are 20 traits. Note that more or less traits and different names of traits are anticipated without veering from the present application.

In the example shown in FIG. 9, a first set of traits 403/404/405/406/407 (five traits in this example) are shown and the user is requested to rank these traits as per the instructions 401: “Rank these traits from the most important at the top.” The instructions 401 also include a description 402 of how the user is to rank the traits: “For you, the most important [trait] is the one, if improved, will have the greatest impact on your life.”

In the first primary user interface 400, the user drags each of the traits 403/404/405/406/407 into the desired ranking position. For example, in the list of the traits 403/404/405/406/407 shown in FIG. 9, the undisciplined trait 403 is at the top of the list, meaning that this is the most important trait (in this list). The most important trait is the one that, if improved, will have the greatest impact on this user's life. If a different one of the first set of traits 403/404/405/406/407 is more important to improve, the user will drag that trait to the top. In some embodiments, the traits 403/404/405/406/407 are dragged by respective anchors 409 or by selecting and holding the trait 403/404/405/406/407 and dragging that trait 403/404/405/406/407 into position. Any user interface is anticipated, including assigning a unique number (e.g., 1 through 5) to each entry of the list.

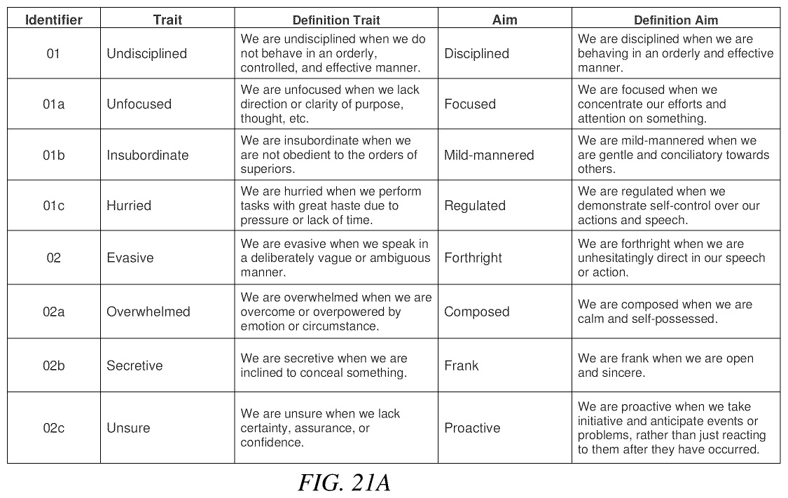

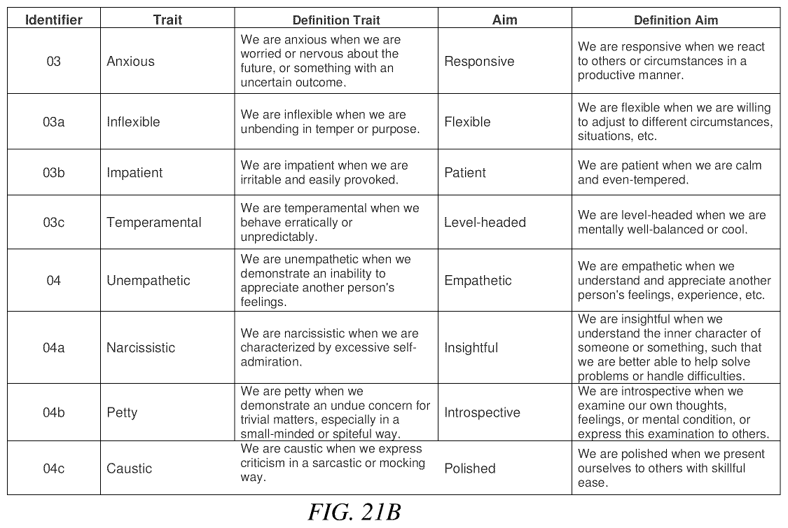

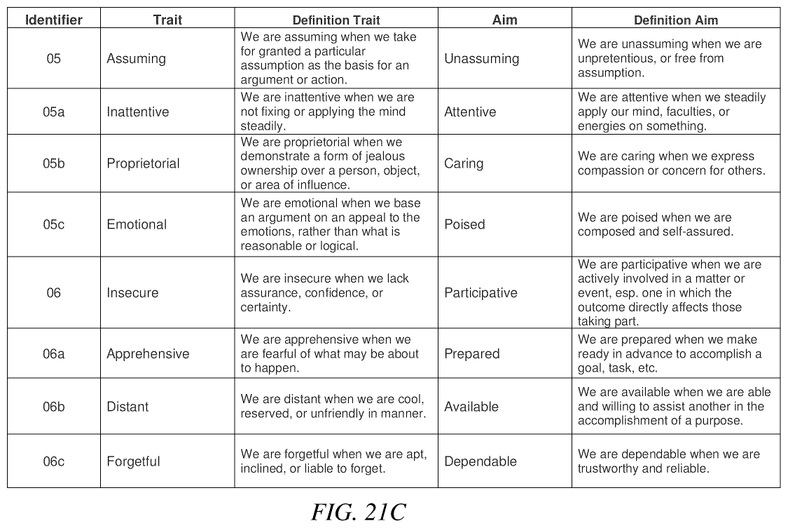

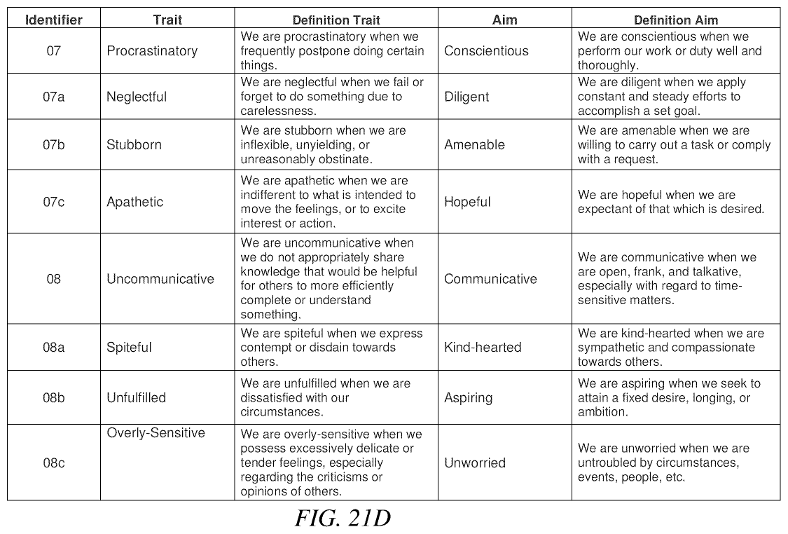

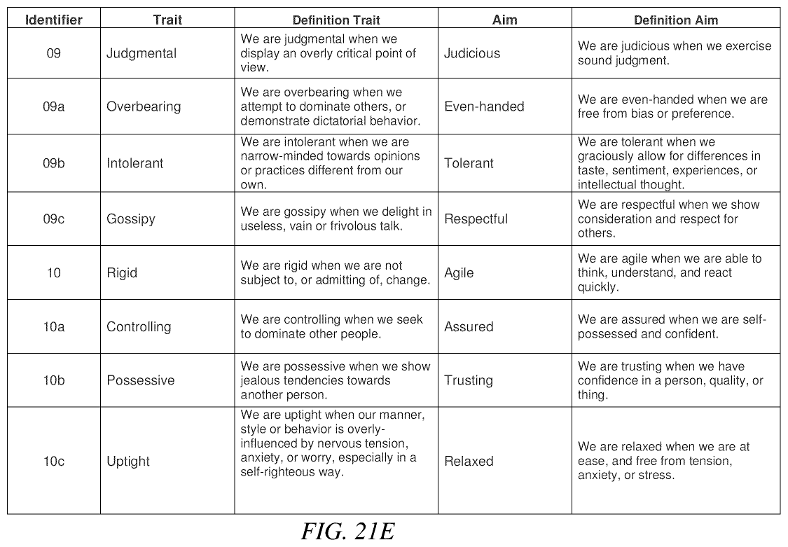

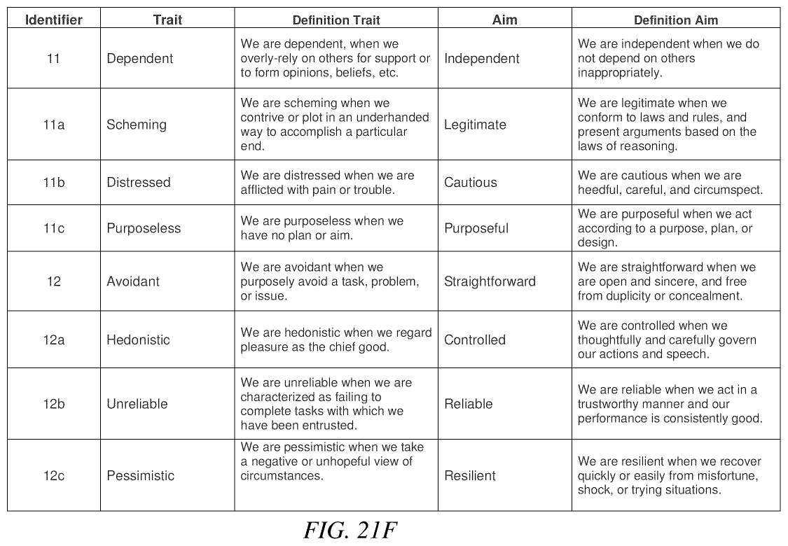

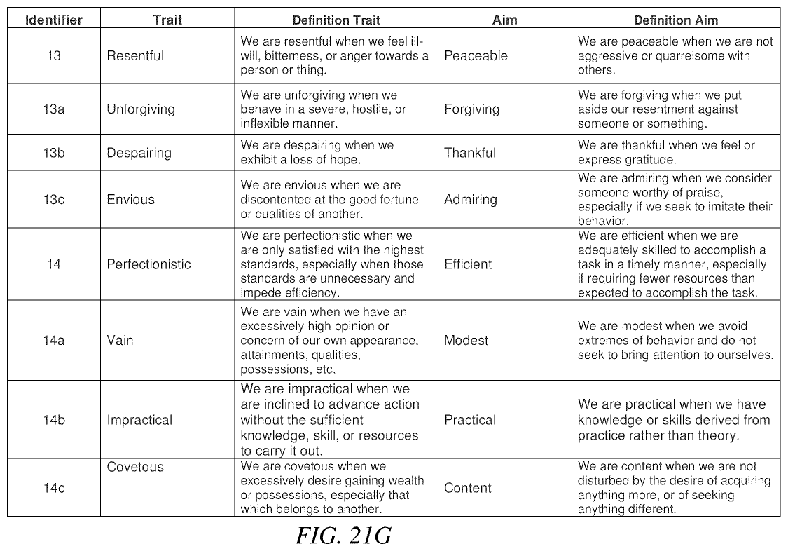

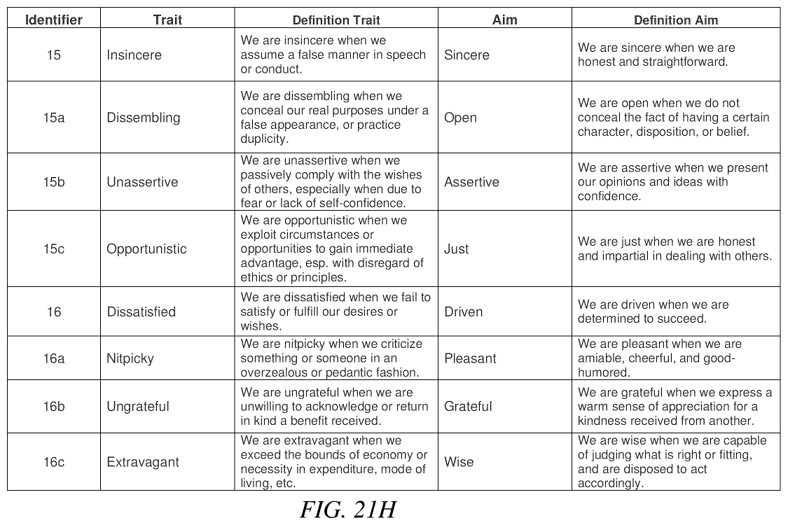

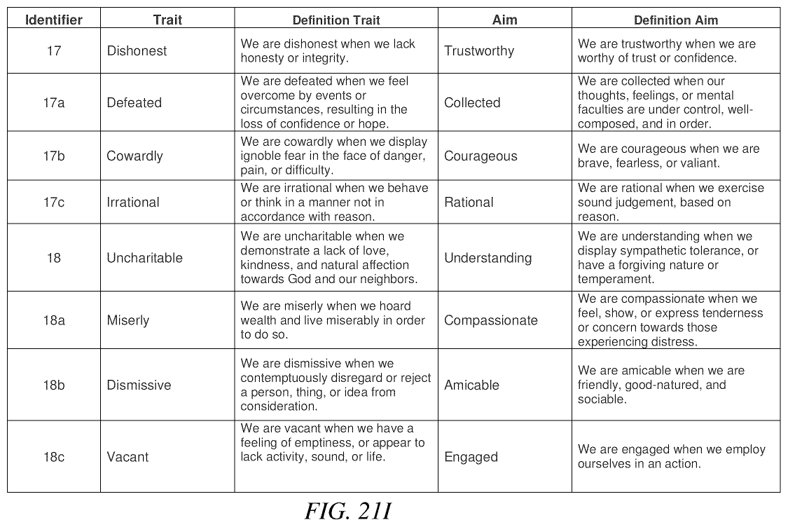

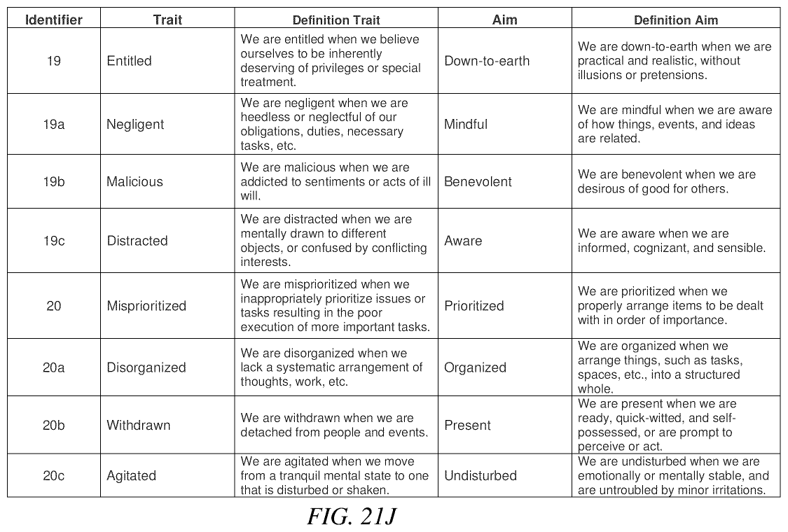

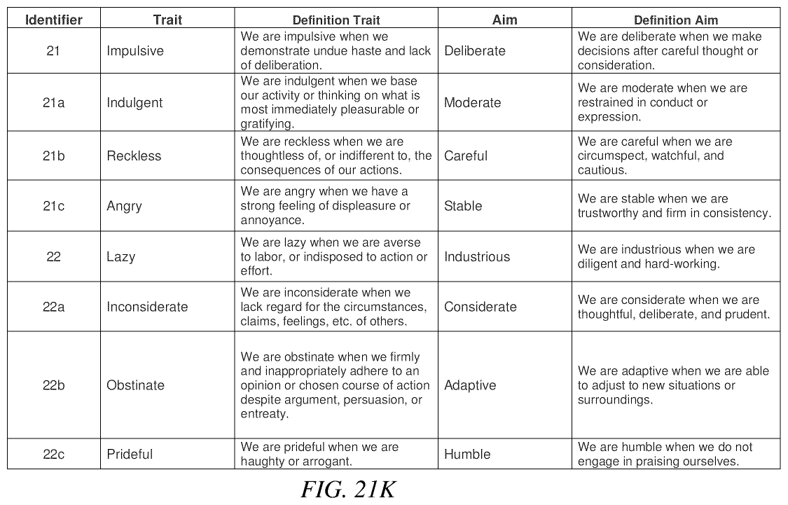

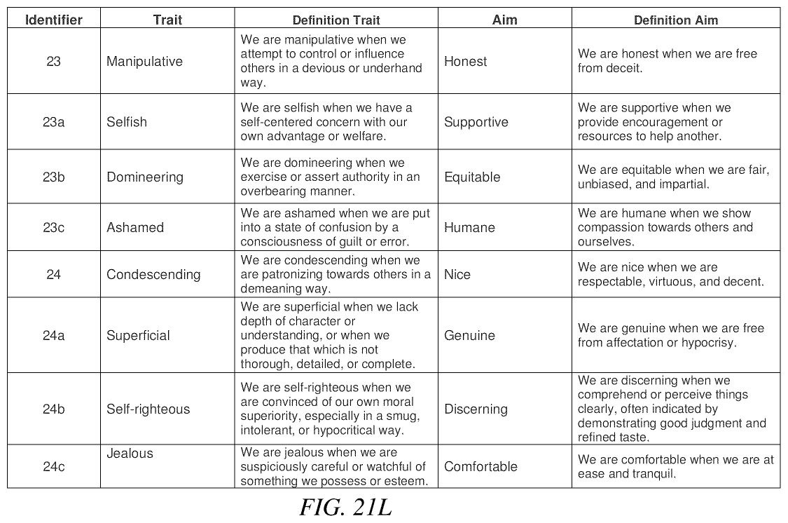

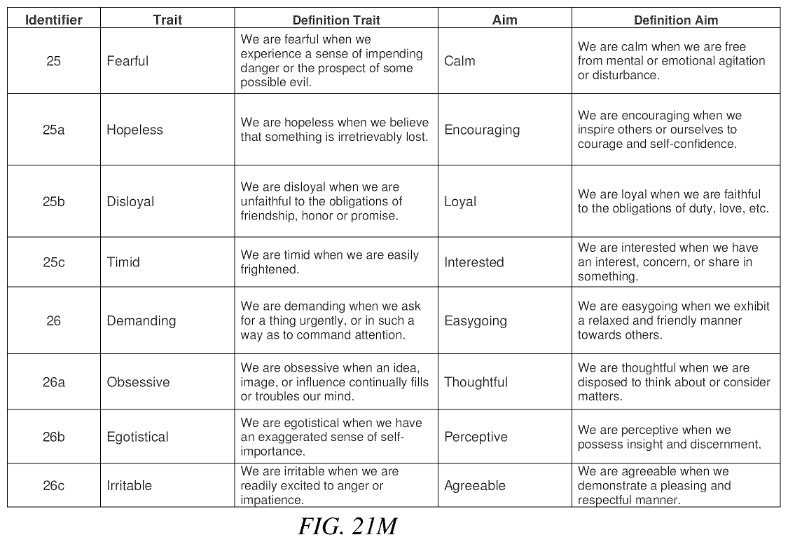

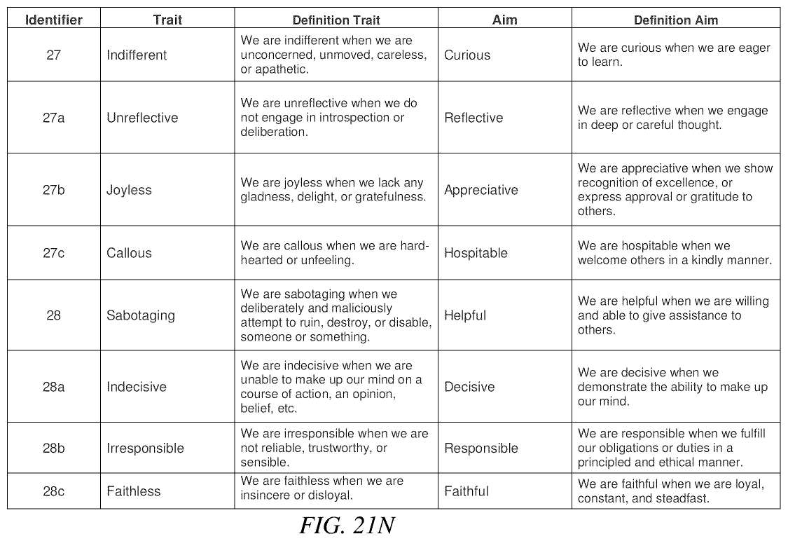

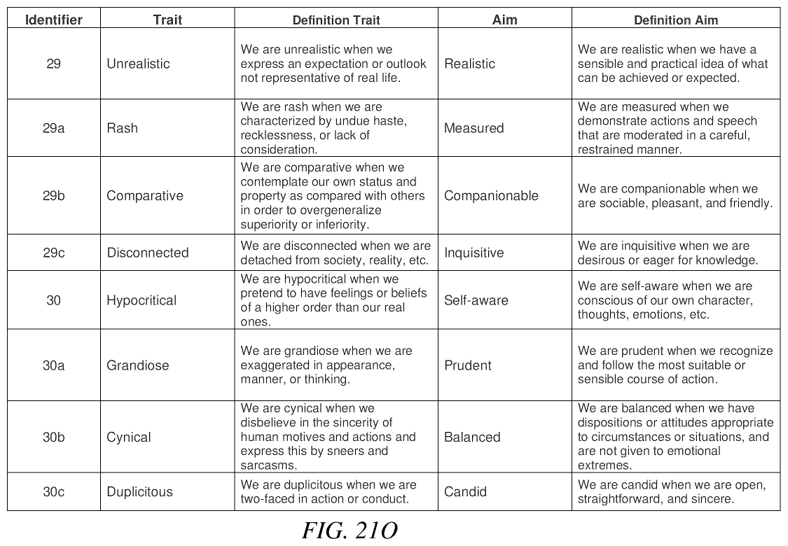

Each of the first set of traits 403/404/405/406/407 also includes an explanation link 411. For example, upon selecting the explanation link 411 for the trait of “undisciplined,” a definition user interface 420 as shown in FIG. 9A is displayed having a detailed description 421 of what it means to be undisciplined. The wording of these descriptions is very important so as to provide the user with a good understanding of what each trait is while not alienating the user. For example, the detailed description 421 for “undisciplined” is worded: “We are undisciplined when we do not behave in an orderly, controlled, and effective manner.” Activation of the explanation link 411 will show a definition of the related trait associated with that explanation link 411. A list of the traits for the personal trait tracker along with definitions of each trait is shown in FIGS. 21A-21O.

In the definition user interface 420, in some embodiments, there is a speaker icon 422, activating such will read out loud the detailed description 421 through an audio output device of the smartphone 10 (or other connected audio device). When the user is finished with the description, invoking the exit icon 423 reverts back to the previous user interface (e.g., whatever primary or secondary user interface came prior).

Again, the example being used is for a personal trait tracker application in which there are thirty (30) identified traits possible. As each of the six primary user interfaces 400/440/460/480/520 allow for the selection from this total of thirty traits, each of the six primary user interfaces 400/420/440/460/480/520 will have five traits that are to be ranked by the user (5*6=30). It should be noted that for other trait tracking applications such as professional and academic, different counts of primary user interfaces are anticipated with more or less traits per primary user interface as needed for the total number of traits in each specific trait tracking applications.

Note at the bottom of the exemplary user interfaces are “goto” buttons 413/414/415/416. For example, the build button 413 is activated to go to the ranking/selection user interfaces, the track button 414 is activated to go to the user interfaces in which the user enters daily activities, the report button 415 is activated to go to the user interfaces for displaying reports, and the account button 416 is activated to go to the user interfaces for displaying and changing account information.

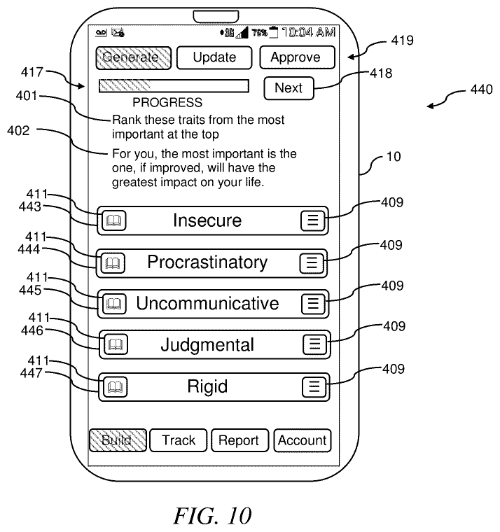

Referring to FIG. 10, a second primary user interface 440 is shown. After invoking the next directive 418 of the first primary user interface 400, the second primary user interface 440 is displayed. In the second primary user interface 440, a second set of traits 443/444/445/446/447 (five traits in this example) are shown and the user is requested to rank these traits as per the instructions 401.″ As before, the instructions 401 also include a description 402 of how the user is to rank the traits.

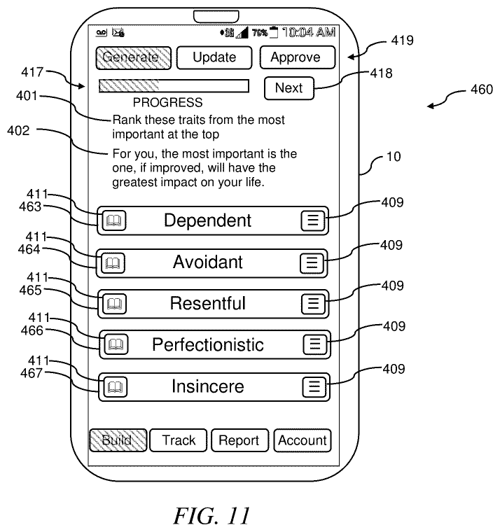

Referring to FIG. 11, a third primary user interface 460 is shown. After invoking the next directive 418 of the second primary user interface 440, the third primary user interface 460 is displayed. In the third primary user interface 460, a third set of traits 463/464/465/466/467 (five traits in this example) are shown and the user is requested to rank these traits as per the instructions 401.″ As before, the instructions 401 also include a description 402 of how the user is to rank the traits.

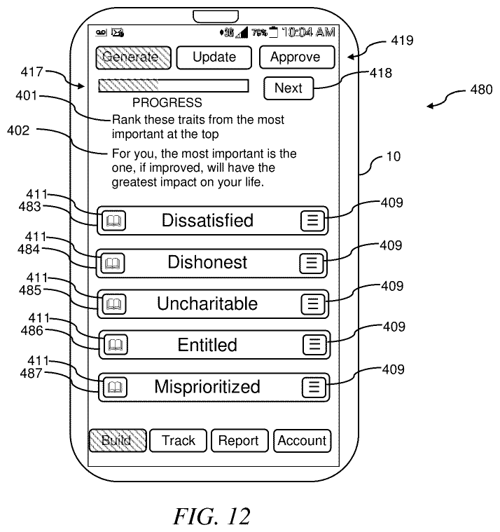

Referring to FIG. 12, a fourth primary user interface 480 is shown. After invoking the next directive 418 of the third primary user interface 460, the fourth primary user interface 480 is displayed. In the fourth primary user interface 480, a fourth set of traits 483/484/485/486/487 (five traits in this example) are shown and the user is requested to rank these traits as per the instructions 401.″ As before, the instructions 401 also include a description 402 of how the user is to rank the traits.

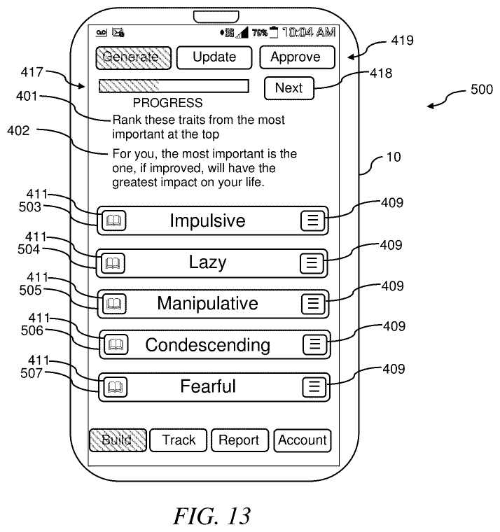

Referring to FIG. 13, a fifth primary user interface 500 is shown. After invoking the next directive 418 of the fourth primary user interface 480, the fifth primary user interface 500 is displayed. In the fifth primary user interface 500, a fifth set of traits 503/504/505/506/507 (five traits in this example) are shown and the user is requested to rank these traits as per the instructions 401.″ As before, the instructions 401 also include a description 402 of how the user is to rank the traits.

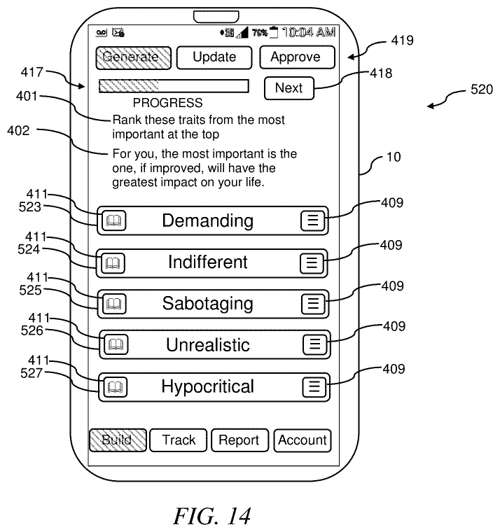

Referring to FIG. 14, a sixth primary user interface 520 is shown. After invoking the next directive 418 of the fifth primary user interface 500, the sixth primary user interface 520 is displayed. In the sixth primary user interface 520, a sixth set of traits 523/524/525/526/527 (five traits in this example) are shown and the user is requested to rank these traits as per the instructions 401.″ As before, the instructions 401 also include a description 402 of how the user is to rank the traits.

After completing the sixth primary user interface 520, the user has been exposed to all 30 traits and has ranked their top 18 traits, three from each of the six sets of 5 traits. In this embodiment (personal development trait tracker), the bottom two traits of each set of traits are removed from consideration, unless the user revisits any of the primary user interfaces 400/420/460/480/500/520 and modifies the order of the traits. This leaves a set of 18 traits for the personal trait tracker that is used as an example in this disclosure. Note in other trait tracker applications, a different or same number of bottom-ranked traits are removed from consideration.

In order to help the user keep in mind the traits deemed most important and not to overwhelm the user, another round of ranking is performed (second phase), this time mixing traits from each list so that each trait that is further ranked in the secondary user interfaces 560/580/600 is combined with traits that were in different lists on the primary user interfaces 400/420/460/480/500/520 to make sure each trait is viewed with all other traits.

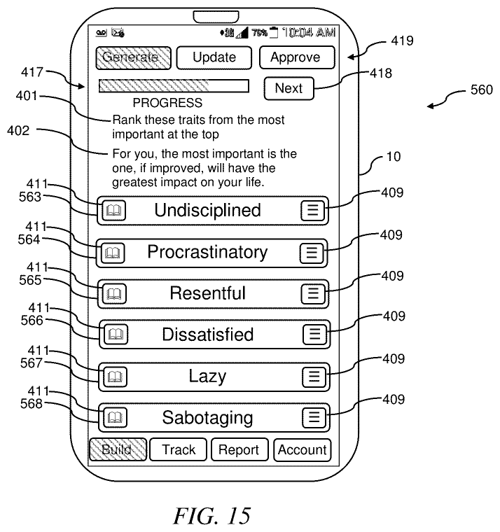

Referring to FIG. 15, an example of combining traits from the top three traits of the primary user interfaces 400/420/460/480/500/520 is shown. In this, the first secondary user interface 560 presents a first secondary set 563/564/564/566/567/568 of traits that include the highest ranked trait (undisciplined trait 403) from the first primary user interface 400, the second highest selected trait 444 from the second primary user interface 440, the third highest selected trait 465 from the third primary user interface 460, the highest selected trait 483 from the fourth primary user interface 480, the second highest selected trait 504 from the fifth primary user interface 500, and the third highest selected trait 525 from the sixth primary user interface 520 as shown in FIG. 15. Again, as above, the user ranks the first secondary set 563/564/564/566/567/568 of traits in a similar rank order, the most important on the top.

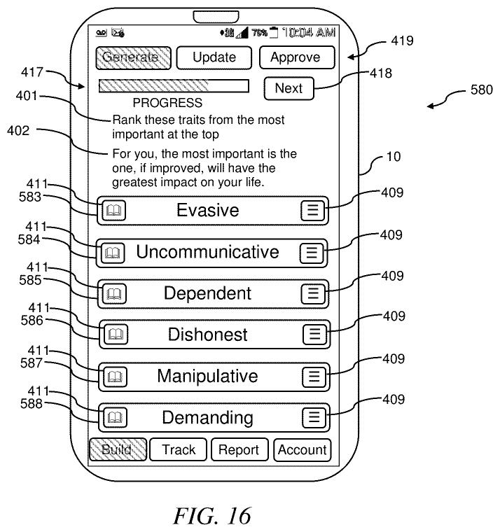

As shown in FIG. 16, the second secondary user interface 580 presents a second secondary set 583/584/584/586/587/588 of traits that include the second highest selected trait 404 from the first primary user interface 400, the third highest selected trait 445 from the second primary user interface 440, the highest selected trait 463 from the third primary user interface 460, the second highest selected trait 484 from the fourth primary user interface 480, the third highest selected trait 505 from the fifth primary user interface 500, and the highest selected trait 523 from the sixth primary user interface 520. Again, as above, the user ranks the second secondary set 583/584/584/586/587/588 of traits in a similar rank order, the most important on the top.

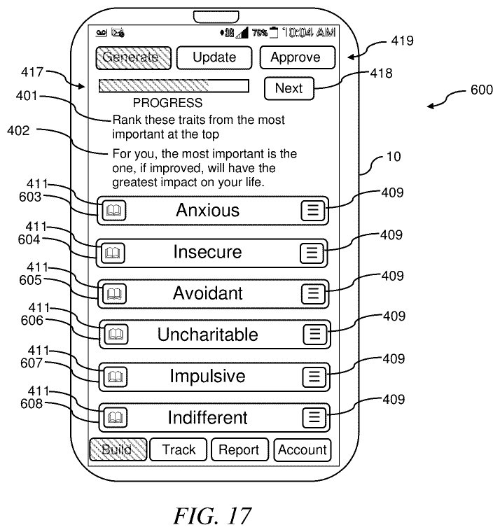

As shown in FIG. 17, the third secondary user interface 600 presents a third secondary set 603/604/604/606/607/608 of traits that include the third highest selected trait 405 from the first primary user interface 400, the highest selected trait 443 from the second primary user interface 440, the second highest selected trait 464 from the third primary user interface 460, the third highest selected trait 485 from the fourth primary user interface 480, the highest selected trait 503 from the fifth primary user interface 500, and the second highest selected trait 524 from the sixth primary user interface 520. Again, as above, the user ranks the third secondary set 603/604/604/606/607/608 of traits in a similar rank order, the most important on the top.

After the user completes the rankings of the first secondary user interface 560, the second secondary user interface 580, and the third secondary user interface 600, in this embodiment, the lowest two traits in each of the first secondary set 563/564/564/566/567/568 of traits, the second secondary set 583/584/584/586/587/588 of traits, and the third secondary set 603/604/604/606/607/608 of traits are removed/ignored/discarded, leaving 12 traits (four from each list).

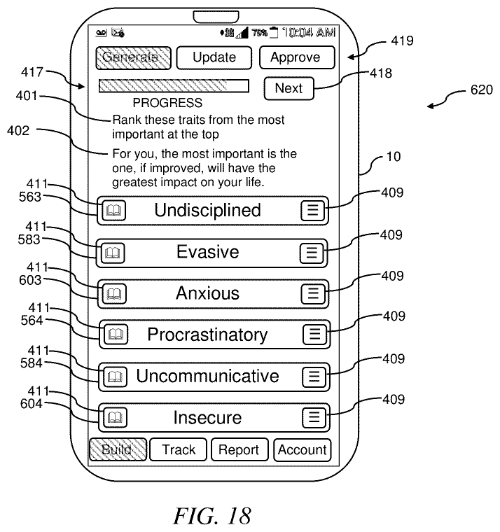

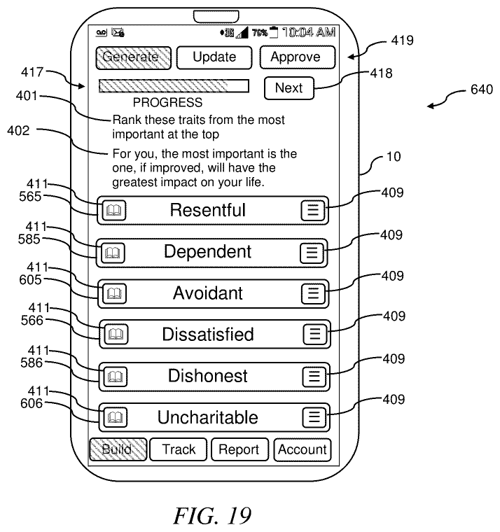

As shown in FIGS. 18 and 19, two final lists are shown in the user interfaces 620/640, each with half of the 12 traits that remain from the first secondary set 563/564/564/566/567/568 of traits, the second secondary set 583/584/584/586/587/588 of traits, and the third secondary set 603/604/604/606/607/608 of traits. Again, the user will rank order each of the two final lists, but this time, no traits are eliminated from the two final lists. Therefore, the user has selected a final set of twelve traits.

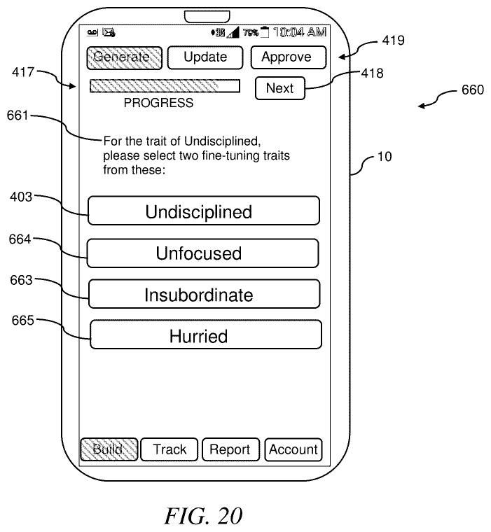

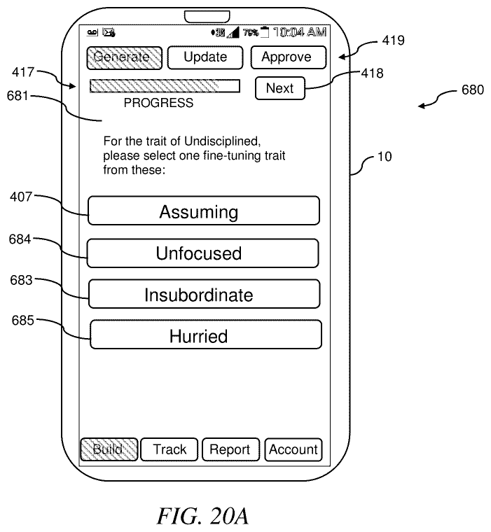

As shown in FIGS. 20 and 20A, the user is now presented with two different fine tuning user interfaces 660/680, For the highest ranked trait in the two final lists, the user is presented with a first fine tuning user interface 660 in which the instructions 661 ask the user to select two fine tuning traits and for the remaining traits in the two final lists, the user is presented with a second fine tuning user interface 680 in which the instructions 681 ask the user to select one fine tuning traits. Examples of the fine tuning traits are shown in FIG. 21A through FIG. 21O. In the identifier column of the traits shown in FIG. 21A through FIG. 21O, the first trait is 01 and the three fine tuning traits are 01a, 01b, and 01c. There are three fine tuning traits defined for each of the original 30 traits. The trait name, definition, aim or goal, and definition of the aim or goal is also shown in FIG. 21A through FIG. 21O.

For each of the highest ranked traits (e.g., the four highest ranked traits), the first fine tuning user interface 660 is presented. The first fine tuning user interface 660 shown has the highest ranked trait (undisciplined 403) along with three fine tuning traits 663/664/665 as taken from the traits and alternate traits of FIG. 21A through FIG. 21O. As the original trait (e.g., undisciplined trait 403) is still present, the user can select two traits from the original trait or fine tuning traits 663/664/665 whichever is more suitable to the user. For each of the highest ranked traits (e.g., the four highest rated traits in the trait tracker for personal improvement) from the final set (e.g., final set of 12 traits for the personal trait tracker), the user is asked to select two fine tuning traits, which can include the original trait) that will go into the working list. This will add four traits to the final working list.

For each of the remaining traits from the final set (e.g., final set of 12 traits for the personal trait tracker), the user selects only one fine-tuning trait only. As shown in FIG. 20A, the second fine tuning user interface 680 shows the fifth highest rated trait, the assuming trait 407 and the user is presented with the assuming trait 407 along with three fine tuning traits 683/684/685 as taken from the traits and alternate traits of FIG. 21A through FIG. 21O. As the original trait (e.g., assuming trait 407) is still present, the user can select the original trait or any fine-tuning traits 683/684/685 whichever is more suitable to the user. For each of the lower ranked traits from the final set (e.g., final set of 12 traits for the personal trait tracker), the user is asked to select one fine-tuning traits (which can include the original trait) that will go into the working list.

Therefore, when the user completes the fine-tuning selection, there are a few more total traits in the working list. For example, for the trait tracking for personal improvement, the user will select two alternate traits for the four highest ranked traits (which can include the original trait) and one alternate trait from the remaining eight traits. This results in a working list of 16 traits.

Referring to FIGS. 21A-21O, a table of traits and aims as used by a personal trait tracker as used in the example user interfaces of FIGS. 9, 9A, and 10-20A is shown. For each row of this table, there is an identification/ID of each line for reference (e.g., 01, 01A), the name of the trait (e.g., “Undisciplined,” “Unfocused”), a definition of the trait (e.g., “We are undisciplined when we do not behave in an orderly, controlled, and effective manner”), an aim for improvement (e.g., one that desires to improve the trait of “Undisciplined” would want to be more “Disciplined”), and a definition of the aim (e.g., “We are disciplined when we are behaving in an orderly and effective manner”).

It should be noted that the definitions that are used are not dictionary definitions. It is asserted that dictionary definitions will not work well as they are often to harsh for someone that is exhibiting a certain trait and wants to improve those traits. For example, the Oxford Languages definition of undisciplined is “lacking in discipline; uncontrolled in behavior or manner.” This is rather curt and standoffish and might offend one who exhibits this trait. Therefore, by carefully wording the definition of the trait in the first-person plural (“we”) to not focus on an issue with the user. Further, the wording is preferably void of harsh, finger-pointing phrases such as “uncontrolled behavior.” It is easier for the user to admit (to themselves) that they are undisciplined if presented in a more neutral, third-person way such as: “We are undisciplined when we do not behave in an orderly, controlled, and effective manner”.

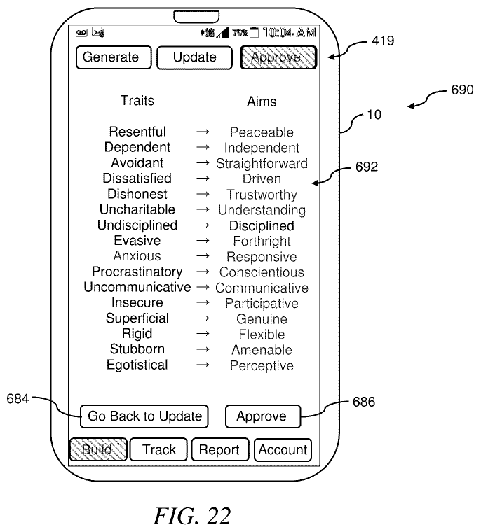

Referring to FIG. 22 illustrates an exemplary smartphone user interface of the system for trait tracking for finalizing a user's list of traits is shown. After ranking is complete, the user is presented with a finalizing user interface 690 as shown in FIG. 22. In this, the user's list of prioritized traits 692 are shown along with aims for each trait. The user has selections to go back 684 (e.g., to make changes to the user's ranking and selected traits) or to approve 686 the user's list of prioritized traits 692. Note that as time goes on, the user will be able to make changes to the user's list of prioritized traits 692 as needed.

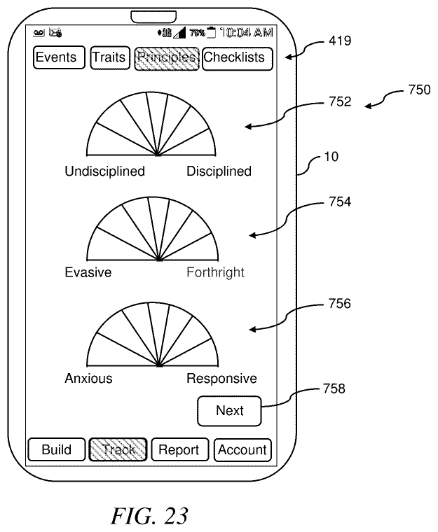

Referring to FIG. 23, an exemplary smartphone user interface of the system for trait tracking for capturing a user's activities for each trait is shown. This daily activity tracker 750 provides selection pies 752/754/756 (note that only three of the user's list of prioritized traits 692 are shown for brevity and clarity reasons) in which the user rates their daily activities with respect to each of the user's list of prioritized traits 692. For example, if one of the traits that the user is trying to improve is “undisciplined” 752, then the user will think back to their day and determine if they were undisciplined for anything during the day and rank themselves based upon their showing of this trait.

The user will select one of the pies based upon how their behavior reflected or improved for each significant trait. Each of their traits is rated on a scale of 1 to 7, where a rating of 1, 2, or 3 (left three pies) represents having been overwhelmed by, significantly impacted by, or just impacted by the significant trait, a rating of 4 (center pie) represents not having been impacted by the significant trait, and a rating of 5 to 7 (right three pies) represents acting in a manner which improves upon the trait, to some extent, to a greater extent, or to an even greater extent. For example, if a user is tracking “Impulsive” as it relates to their shopping habits, and they did not buy something that day, but it never even occurred to them to purchase anything, they would give themselves a 4 (middle pie). If they refrained from making a minor purchase, they would give themselves a 5 (pie right of the middle pie). If they overcame multiple instances of wanting to purchase something more expensive, they may give themselves a 6 or 7 (pies to the right). If they ended up succumbing to a purchase and it was something minor, they might give themselves a 3 (pie left of the middle pie). If they spent more than they should have or more than they can afford, they might give themselves a 2 or 1 (pies at the left). It is fully anticipated that the scale includes more or less than 7 ratings and utilizes any type of input paradigm such as an arrow, a moving icon on a line, a numeric value, etc.

When done rating each trait, the use selects the “next” icon 758.

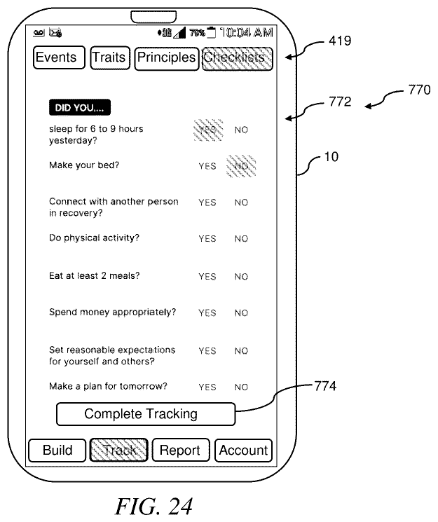

Referring to FIG. 24 illustrates an exemplary smartphone user interface of the system for trait tracking for daily reporting is shown. The daily reporting interface 770 captures information about the user's day through a series of question 772 that will help the user, possibly, understand what led to successes and/or failures with achieving progress for each of the user's list of prioritized traits 692. The example shown in FIG. 24 is more for the recovery embodiment of the trait tracking application, having questions about activities that may or may not lead to the person faltering with improving one or more on the user's list of prioritized traits 692. For example, if one on the user's list of prioritized traits 692 is “avoidant” and the user ranked low on this trait, then the user indicates they did not try to connect with another recovery person, the user will see the cause and effect and try to improve the next day. Note that the series of questions 772 is adjustable and will vary based upon the user and type of trait tracking application being used. The user selects “complete tracking” 774 when finished entering their daily status.

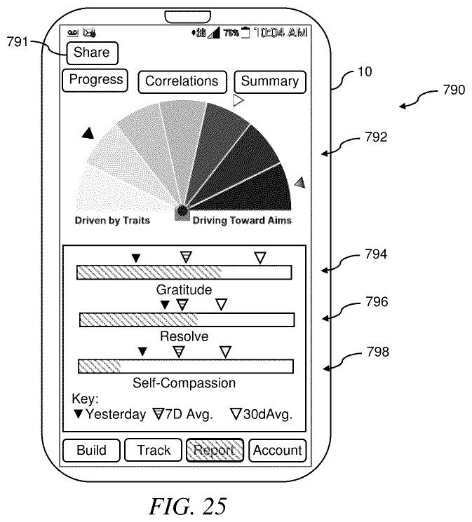

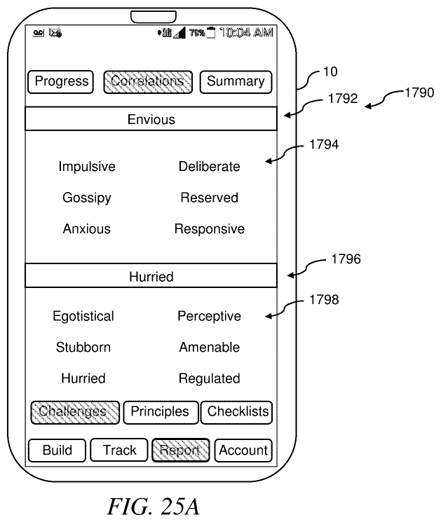

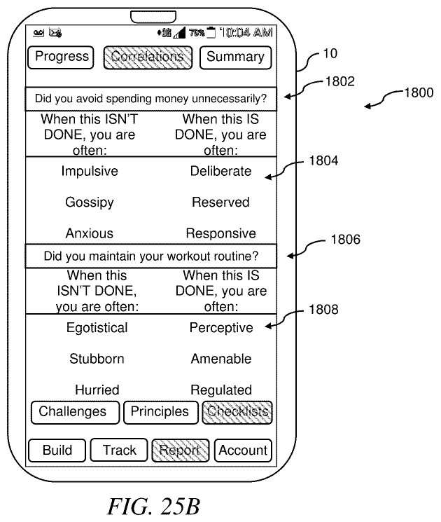

Referring to FIG. 25, an exemplary smartphone user interface of the system for trait tracking for reporting progress to the user is shown. In order to receive feedback, the user has access to one or more reporting user interfaces 790 (one example is shown in FIG. 25, though others are anticipated). The reporting user interfaces 790 in this example shows a graphic chart 792 of the user's progress towards their aims compared to yesterday, compared to the last 7 days, and compared to the last 30 days, though any number of days, weeks, months, or years is anticipated in other variations. Then, for each of the three principles of gratitude 794, resolve 796, and self-compassion 798, individual progress is shown compared to yesterday, compared to the last 7 days, and compared to the last 30 days, though any number of days, weeks, months, or years is anticipated.

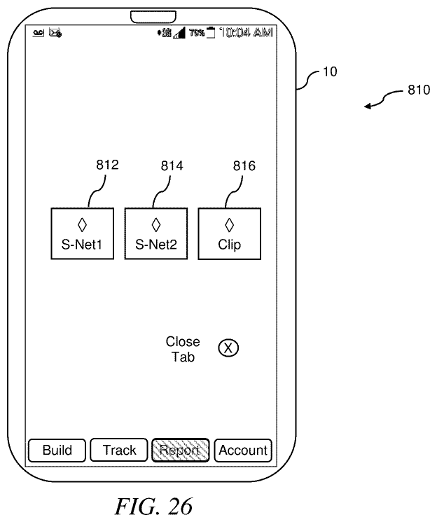

As many users will work with a partner such as another user who is working on improvement, a counselor, a significant other, etc., it is often useful to share the user's progress with another. It should be noted that information about the user is kept confidential through all means known in the industry, including data protection by encryption, etc. If the user wants to share some or all of the user's progress with another, a share operative 791 is provided that invokes the share user interface 810 (see FIG. 26).

Referring to FIG. 25A, an exemplary smartphone user interface of the system for trait tracking for showing correlations 1790 to the user is shown. Although two traits 1792/1796 are shown, by scrolling up/down, all traits are shown. The correlated traits 1794/1798 for the traits 1792/1796 are shown, for example a person that is envious is often impulsive, gossipy, and/or anxious and the desired trait for each is shown, for example, deliberate, reserved and/or responsive.

Referring to FIG. 25B, an exemplary smartphone user interface of the system for trait tracking for showing a correlation check list 1800 to the user. Although two correlations 1802/1806 are shown, by scrolling up/down, all correlations are accessible. The correlated traits 1804/1808 for the correlations 1802/1806 are shown, for example, if the user did not avoid spending money, this is correlated to traits 1804 that include impulsive, gossipy, and/or anxious. If the user did avoid spending money, this is correlated to traits 1804 that include deliberate, reserved and/or responsive.

Referring to FIG. 26, an exemplary smartphone user interface of the system for trait tracking for sharing progress with others is shown. The share user interface 810 provides the ability for the user to share the user's progress with another by way of, for example, any of the popular social networks 812/814, or to clip a copy of the user's progress 816 and share the clipped version by way of email, text message, etc.

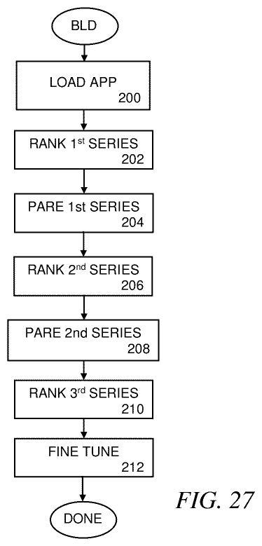

Referring to FIG. 27, an exemplary program flow of the system for trait tracking for ranking traits by a user is shown. In this, the user loads 200 the trait tracking application and after login to the trait tracking application, the user progresses through the first phase, ranking 202 the first series of the traits. Next, after ranking 202 the first series of the traits, the first series of traits is pared 204, for example, removing the bottom two traits from each ranked sub-list as described above.

Next, the user progresses through the second phase, ranking 206 the second series of the traits. After ranking 206 the secondary series of the traits, the list of traits is again pared 208, for example, removing the bottom two traits from each ranked sub-list as described above.

Next, the user progresses through the third phase, ranking 210 a tertiary series of the traits. In this, the list is not pared any further, but for each entry in the remaining list of traits, the user is presented with fine tuning 212 options for each trait in the remaining list of traits to create the final list of traits for that user. Note that this fine tuning provides different words that are similar to each trait in the remaining list for the user to either replace the selected word or add an additional trait to the final list of traits. For example, if the final list of traits has 12 total traits, in some embodiments, when fine tuning, the user selects two fine tuning traits for the top four highest ranked traits and one fine tuning trait for the remaining eight traits, expanding the final list of traits to 16 traits.

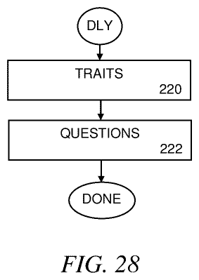

Referring to FIG. 28, an exemplary program flow of the system for trait tracking for daily reporting is shown. Daily reporting includes traits 220 (e.g., self-rating on progress towards each individual aim) and answering a series of questions 222 (e.g., did you get 6-8 hours of sleep last night?).

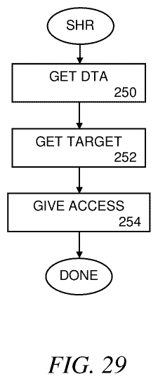

Referring to FIG. 29, an exemplary program flow of the system for trait tracking for sharing progress is shown. As the user's data is securely stored, others will not have access to the user's data (e.g., the user's list of prioritized traits 692 and progress history). When the user provides access to the user's data, the user's data is obtained 250 (e.g., decrypted), the target for the data is determined 252 (e.g., an email address, social network identifier) and the selected user's data is provided 254 to the target. For example, the image of FIG. 25 and/or the data provided in FIG. 25.

Note that in some embodiments, the user's data is provided 254 on a one-time basis while in other embodiments, the user's data is provided 254 on a continuous basis.

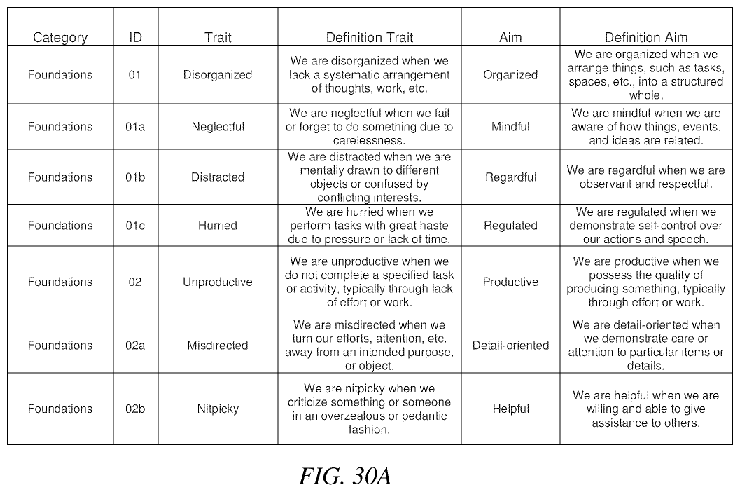

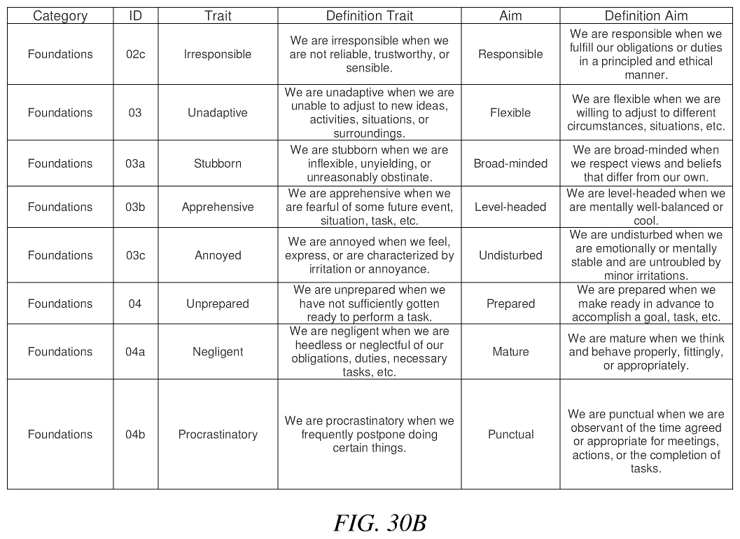

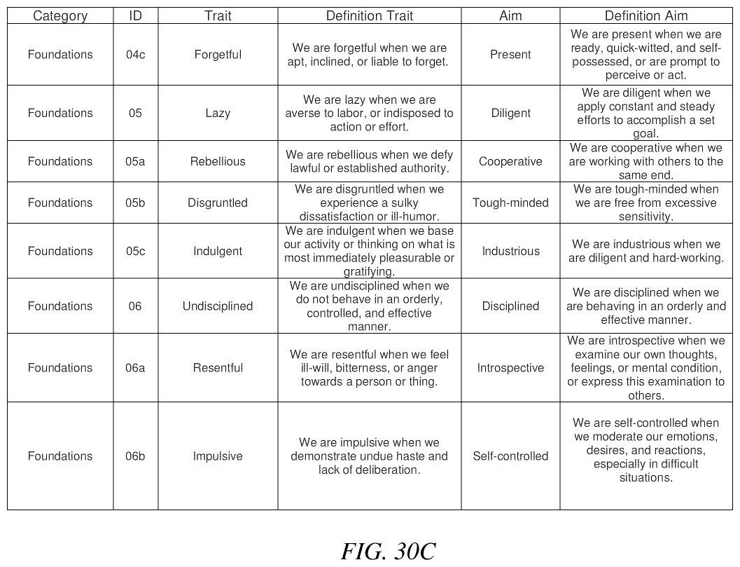

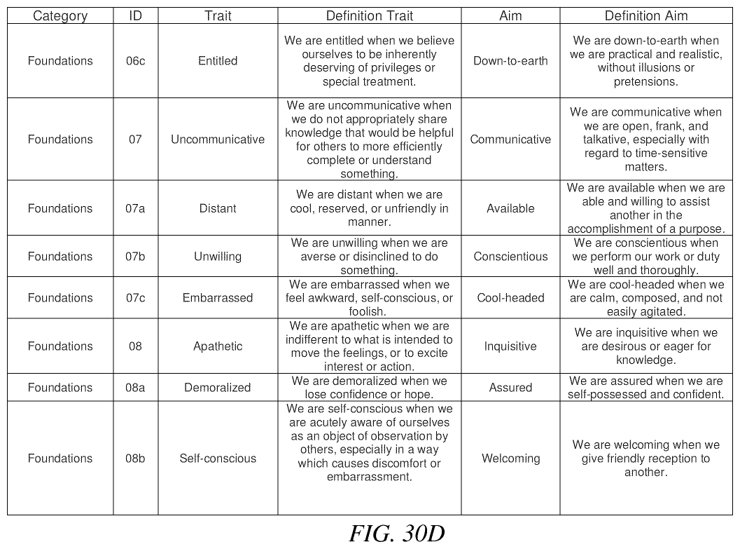

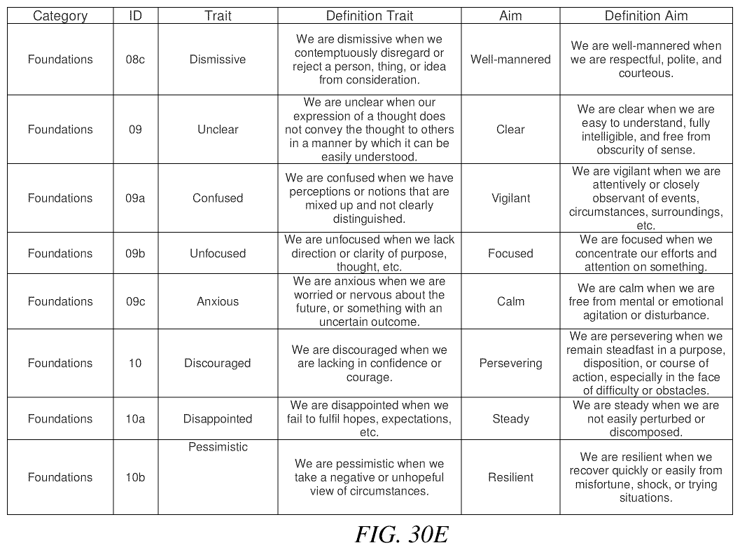

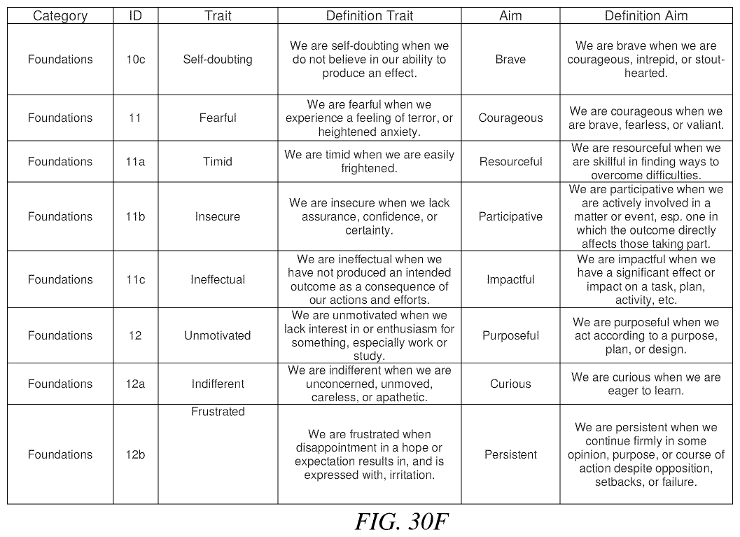

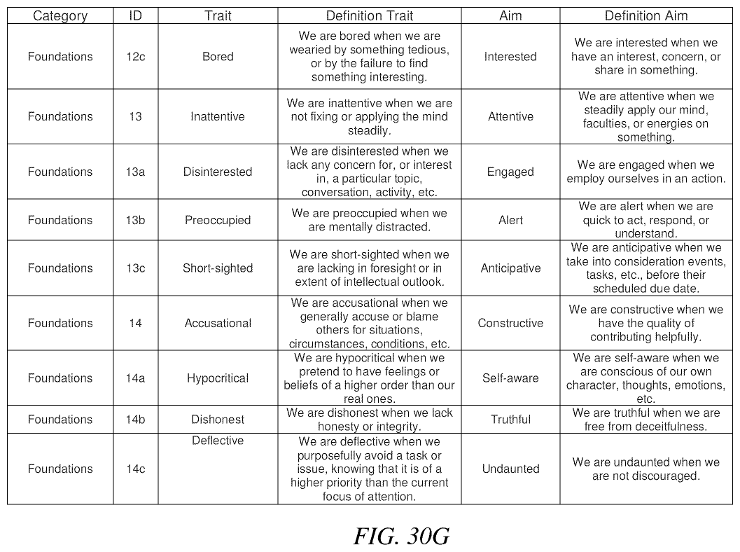

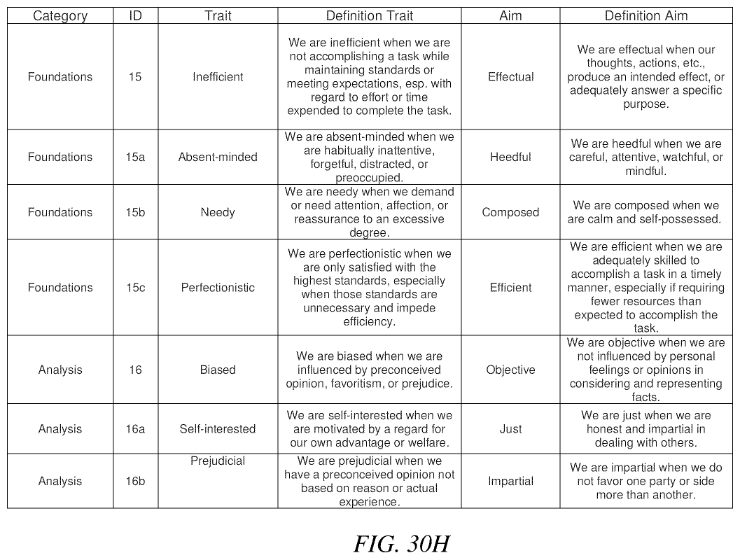

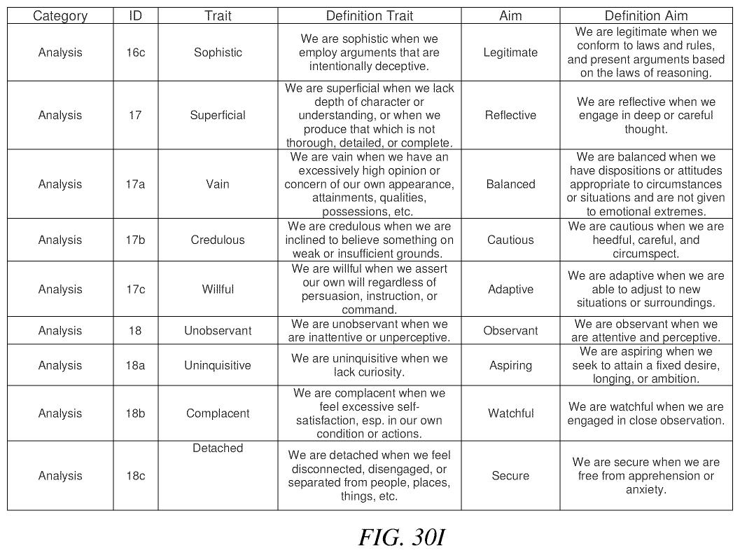

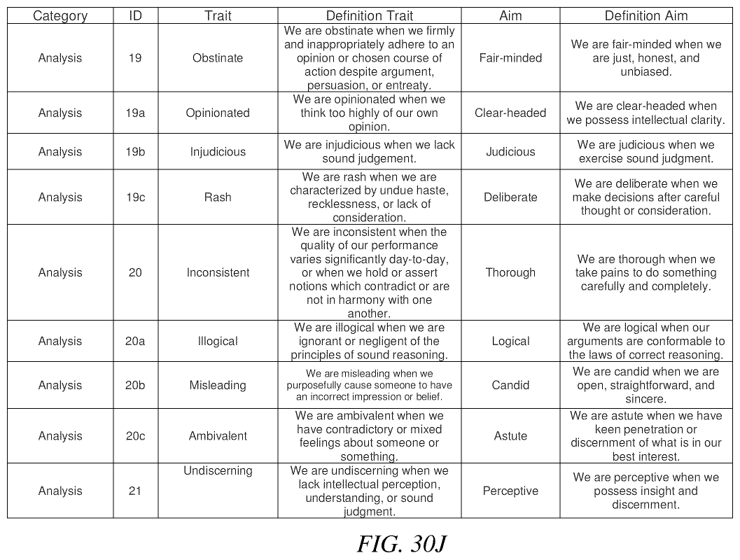

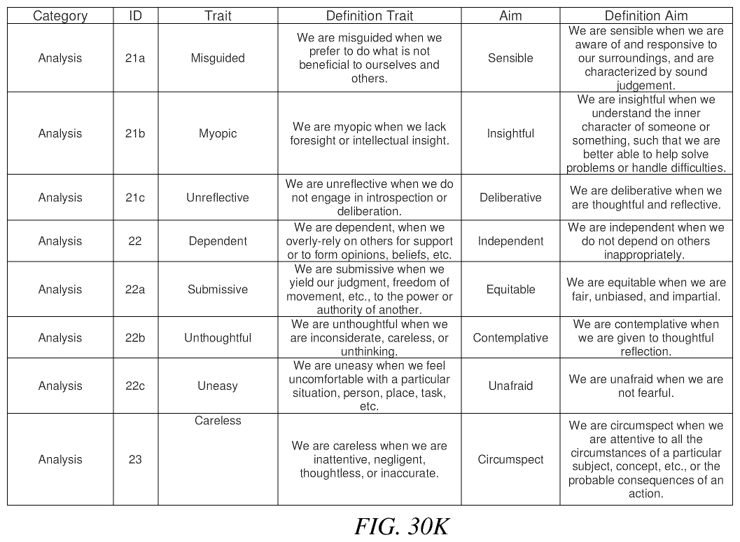

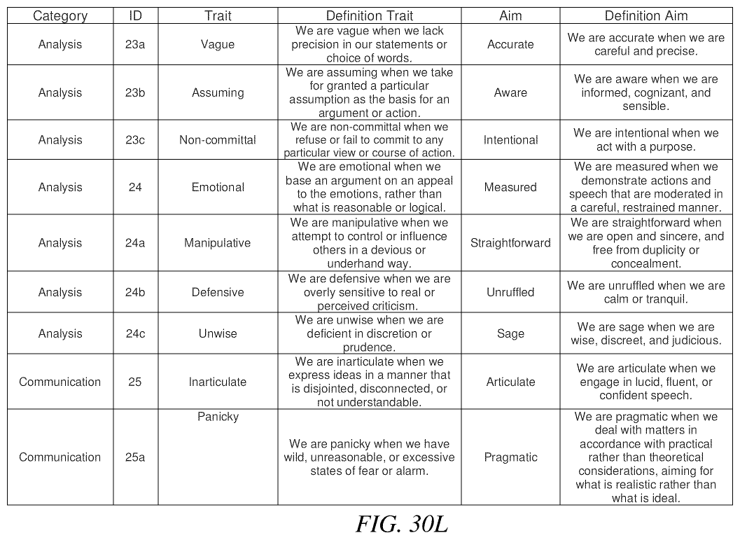

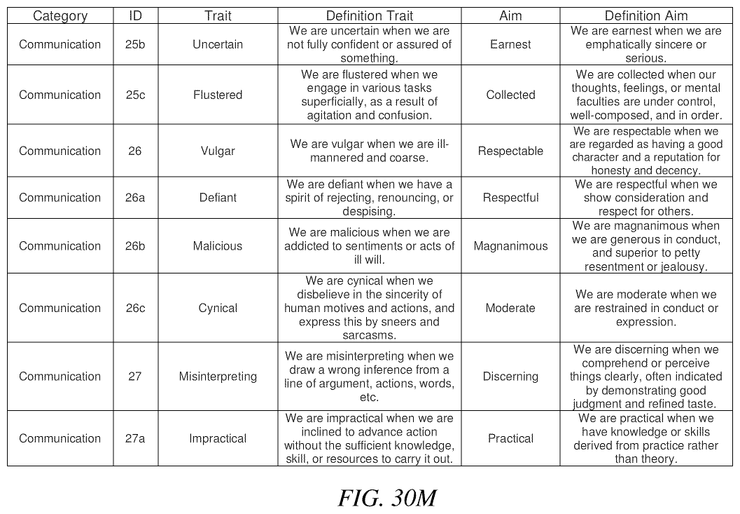

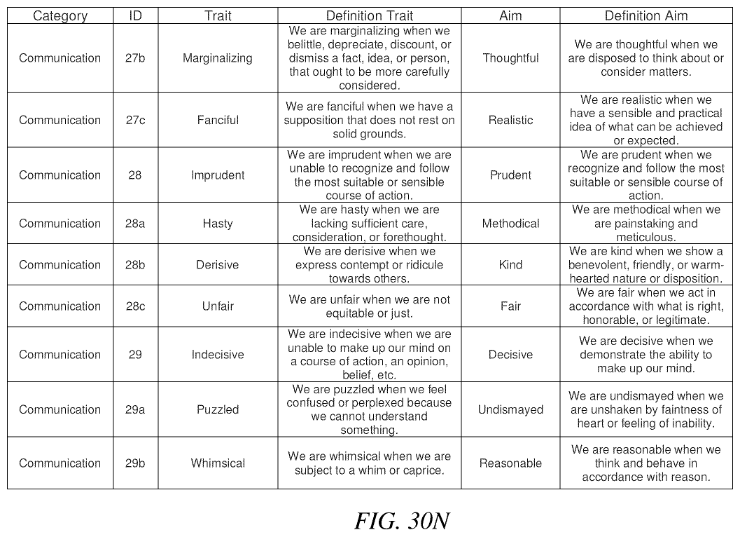

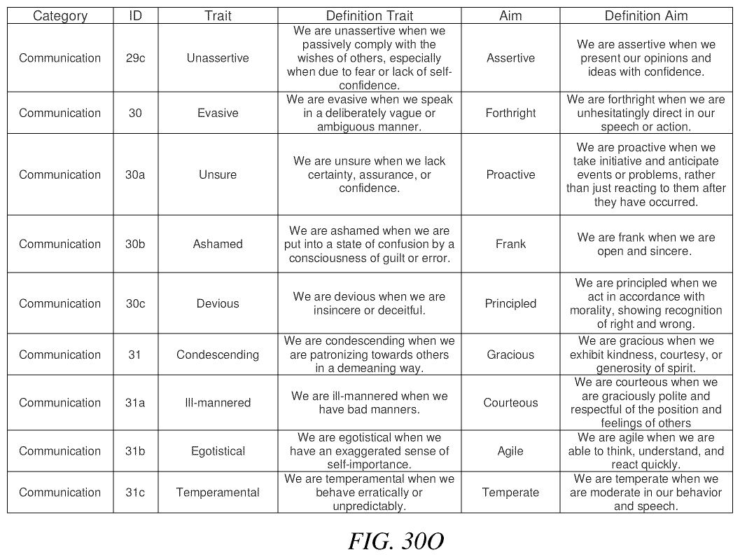

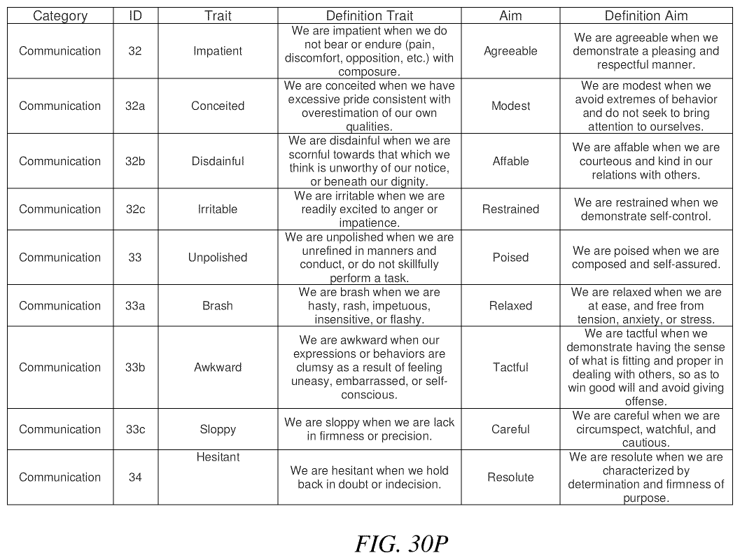

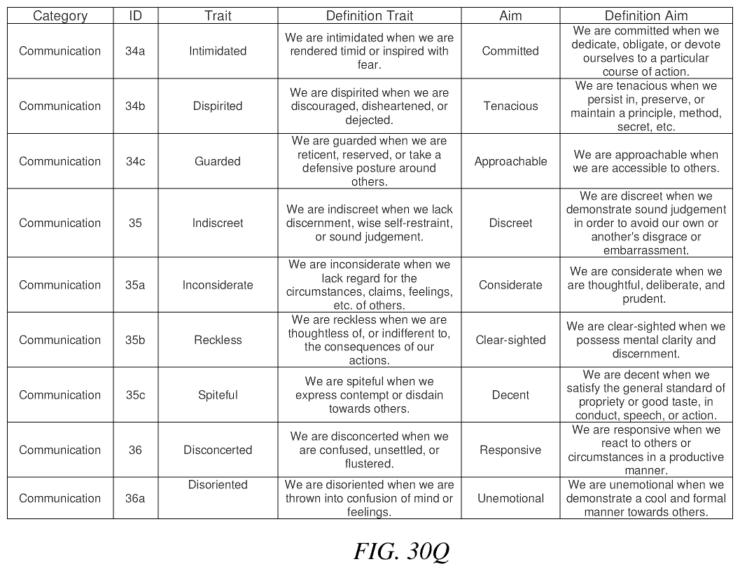

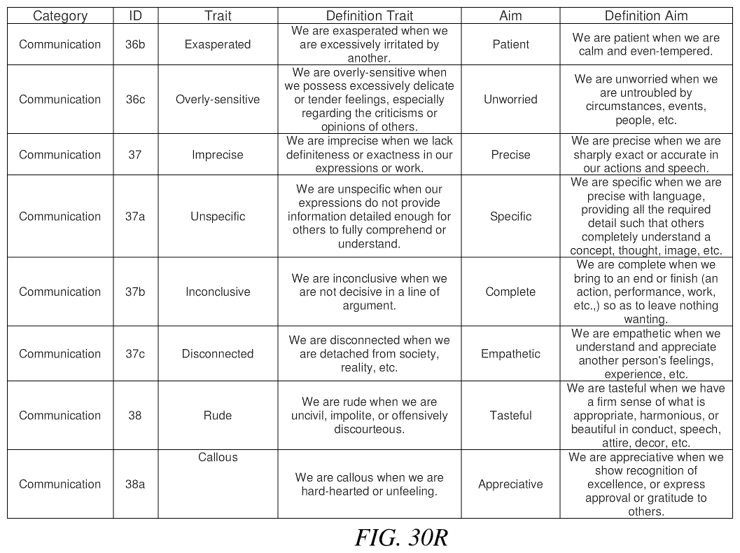

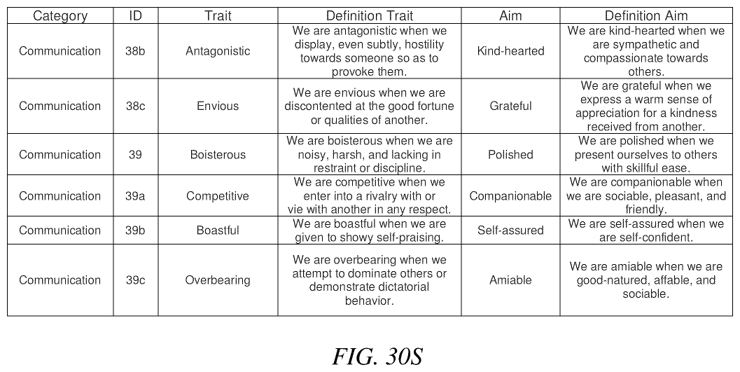

Referring to FIGS. 30A-30S, a table of traits and aims as used by an academic trait tracker application is shown. For each row of this table, there is an identification/ID of each line for reference (e.g., 01, 01A), the name of the trait (e.g., “Undisciplined,” “Unfocused”), a definition of the trait (e.g., “We are undisciplined when we do not behave in an orderly, controlled, and effective manner”), an aim for improvement (e.g., one that desires to improve the trait of “Undisciplined” would want to be more “Disciplined”), and a definition of the aim (e.g., “We are disciplined when we are behaving in an orderly and effective manner”). For the academic trait tracking application, there is an additional category field to sub-divide the traits into “foundations,” “analysis,” and “communications.”

Again, it should be noted that the definitions that are used are not dictionary definitions. It is asserted that dictionary definitions will not work well as they are often too harsh for someone that is exhibiting a certain trait and wants to improve those traits.

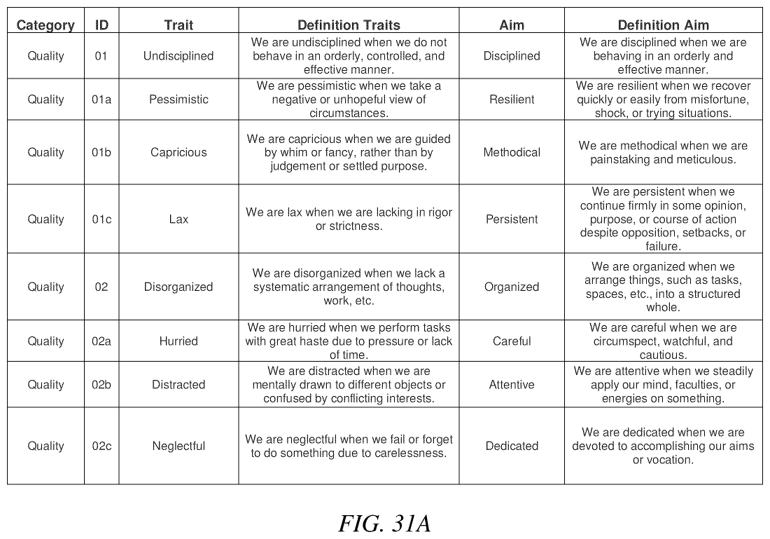

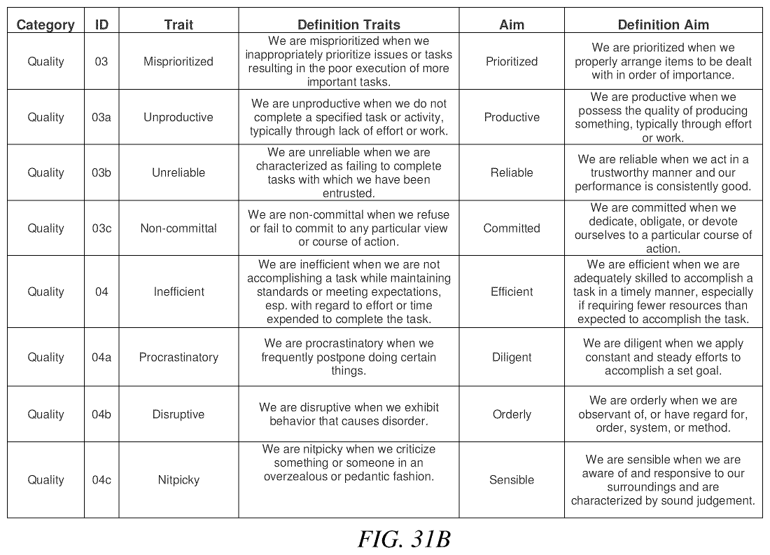

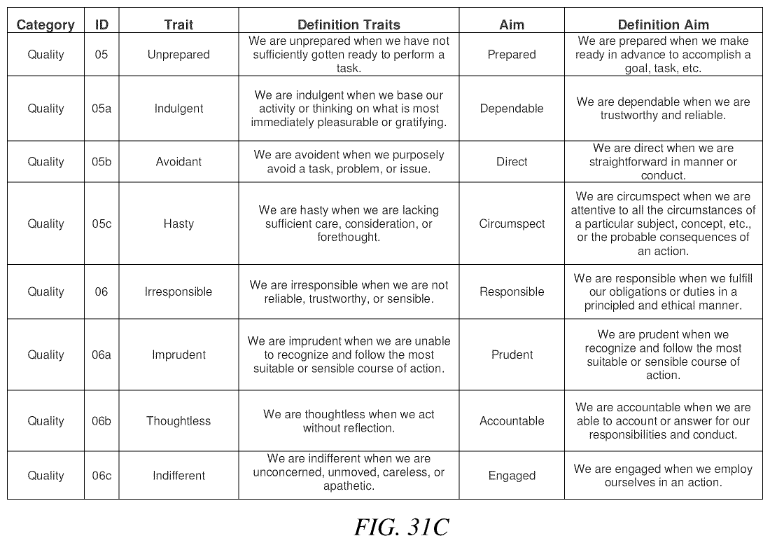

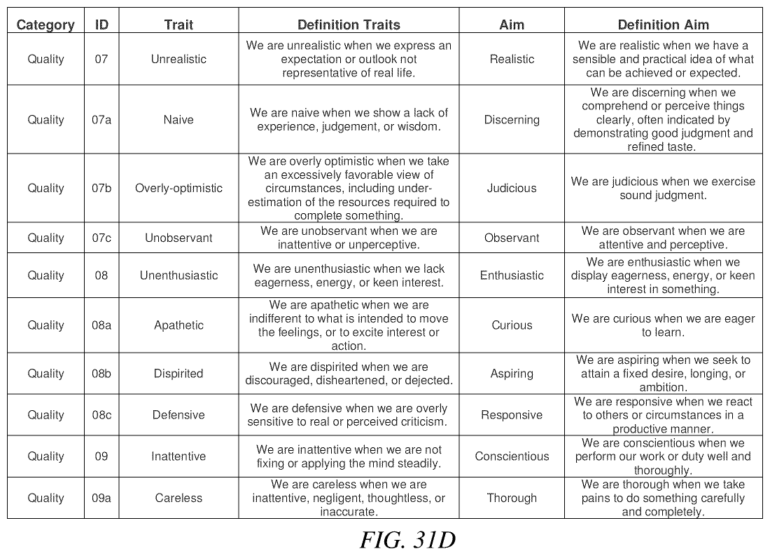

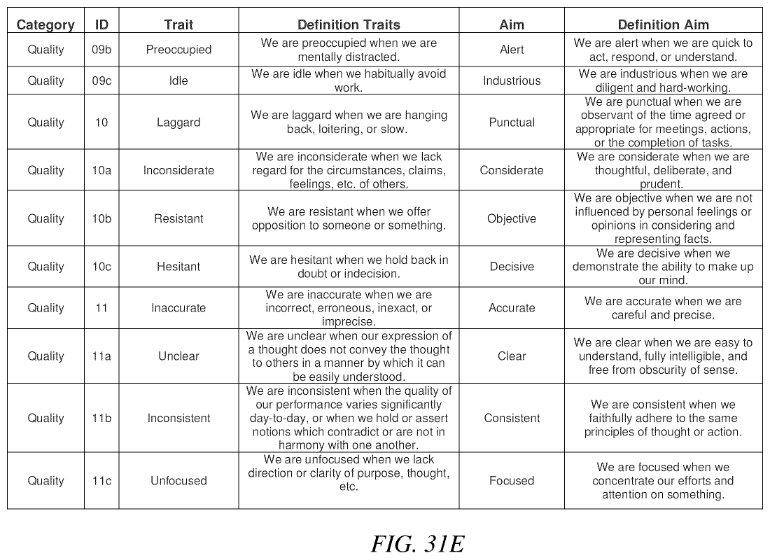

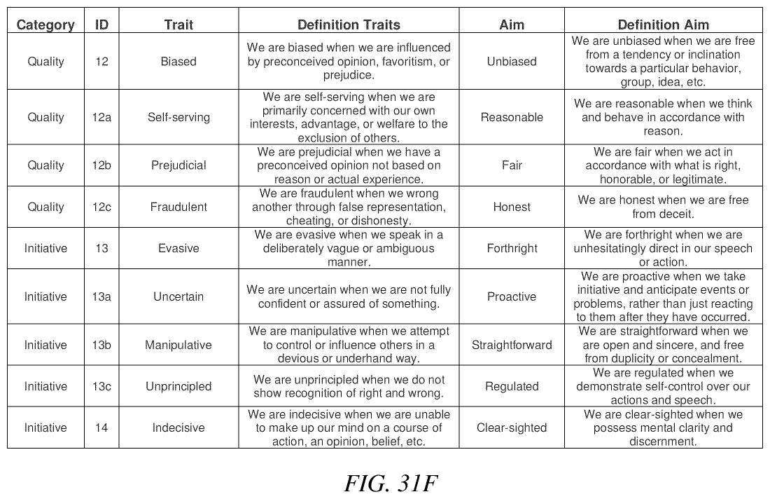

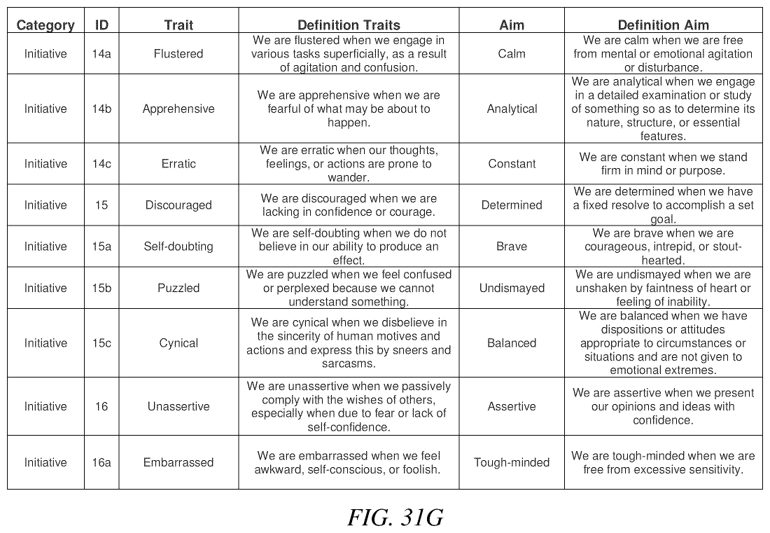

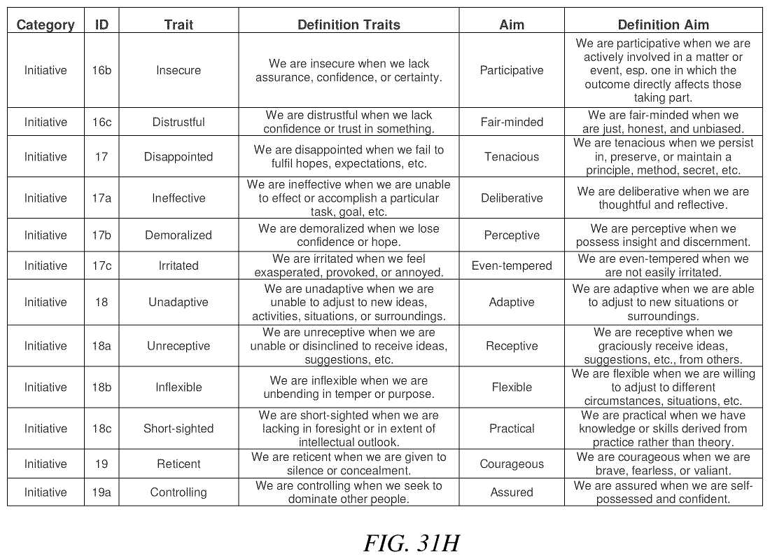

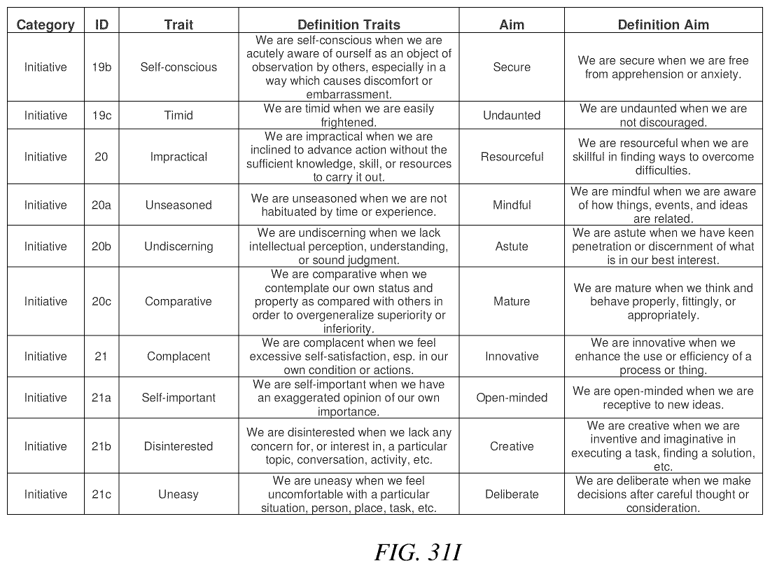

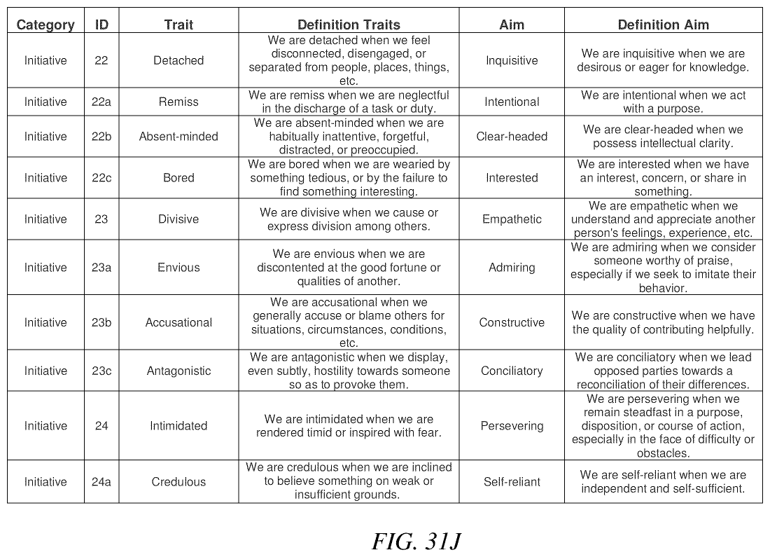

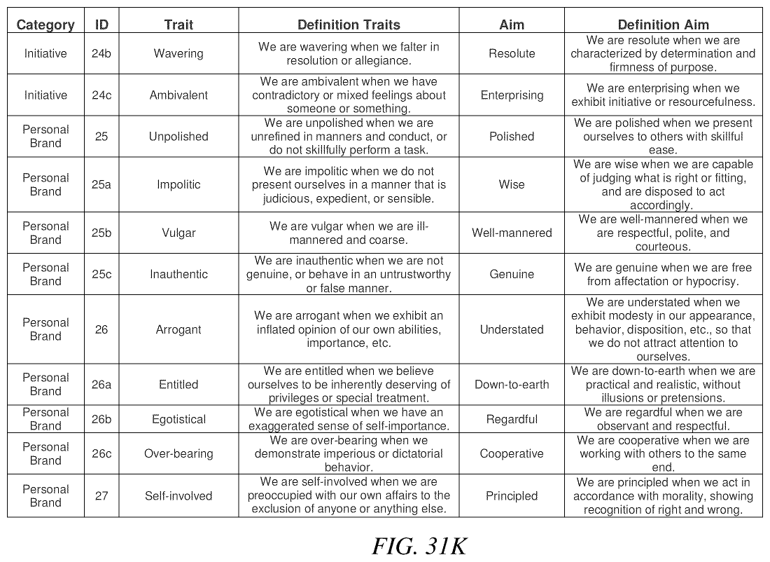

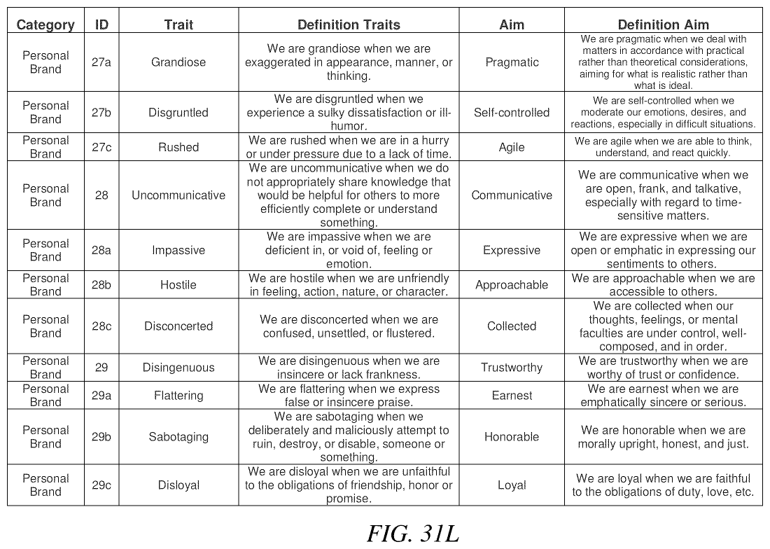

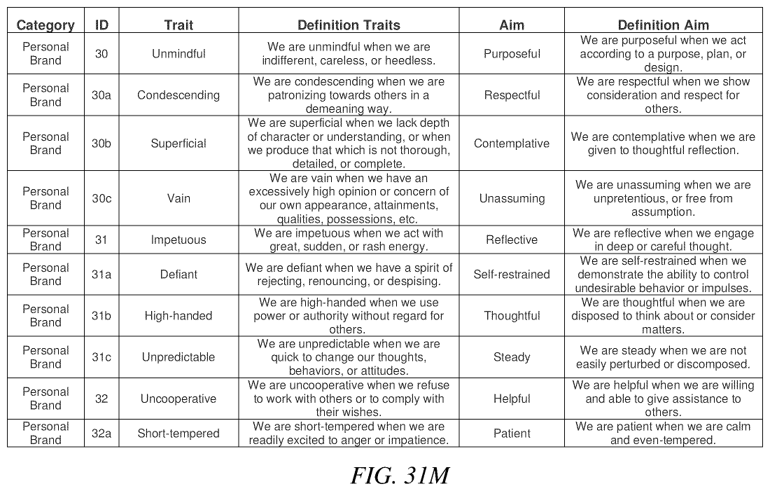

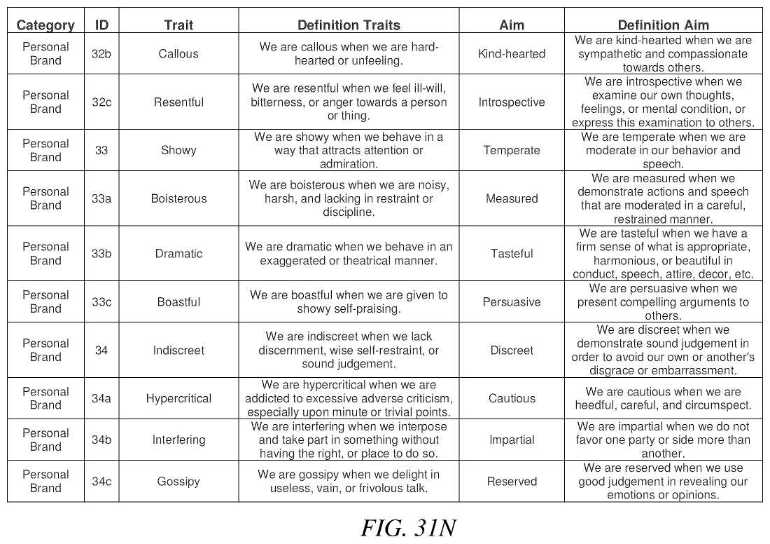

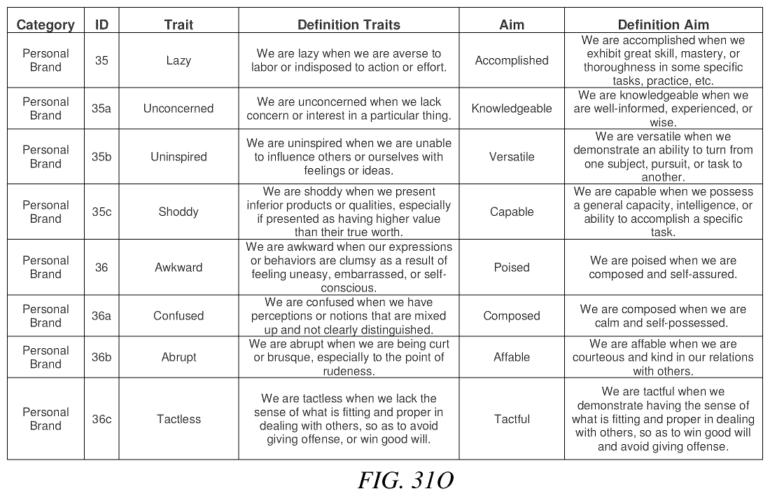

Referring to FIGS. 31A-31O, a table of traits and aims as used by a professional trait tracker application is shown. For each row of this table, there is an identification/ID of each line for reference (e.g., 01, 01A), the name of the trait (e.g., “Undisciplined,” “Unfocused”), a definition of the trait (e.g., “We are undisciplined when we do not behave in an orderly, controlled, and effective manner”), an aim for improvement (e.g., one that desires to improve the trait of “Undisciplined” would want to be more “Disciplined”), and a definition of the aim (e.g., “We are disciplined when we are behaving in an orderly and effective manner”). For the professional trait tracking application, there is an additional category field to sub-divide the traits into “quality,” “initiative,” and “personal brand.”

Again, it should be noted that the definitions that are used are not dictionary definitions. It is asserted that dictionary definitions will not work well as they are often to harsh for someone that is exhibiting a certain trait and wants to improve those traits.

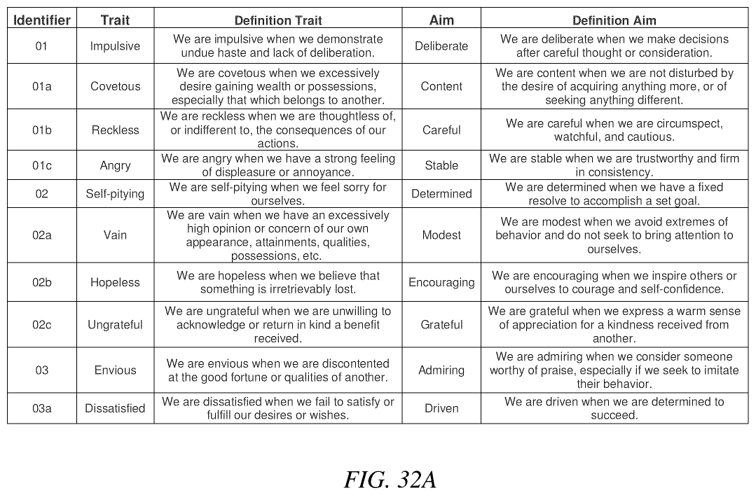

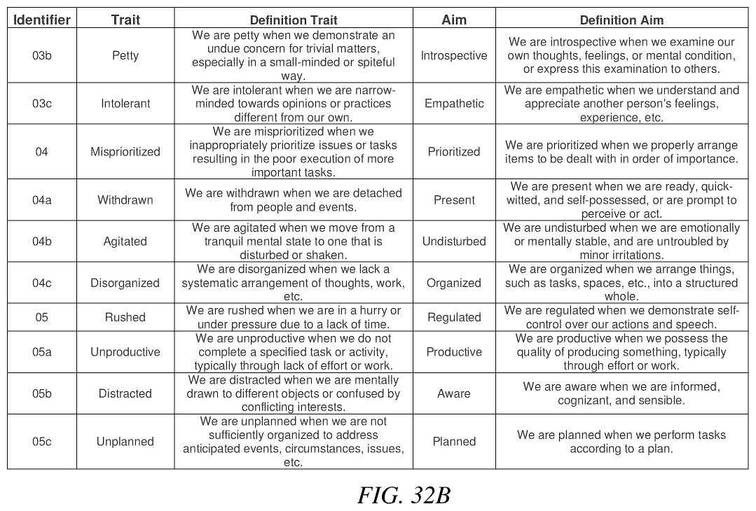

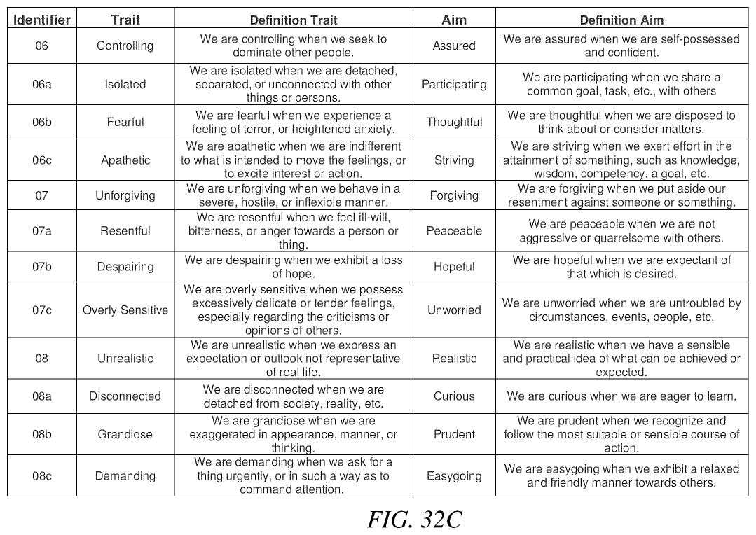

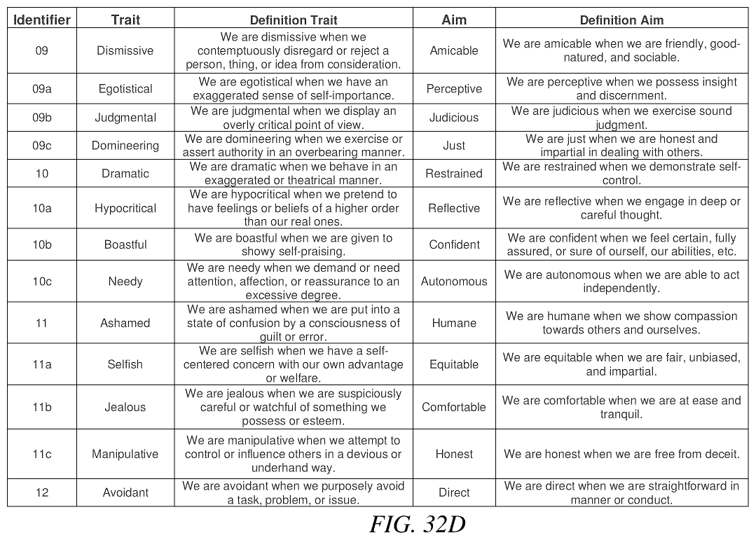

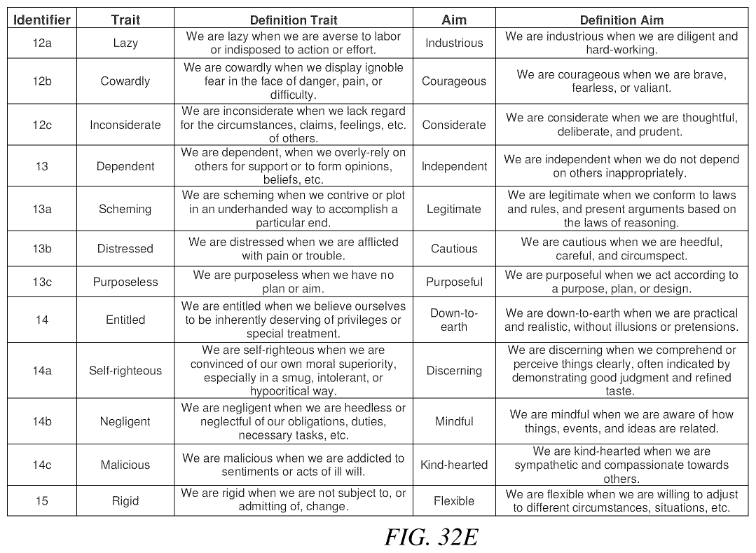

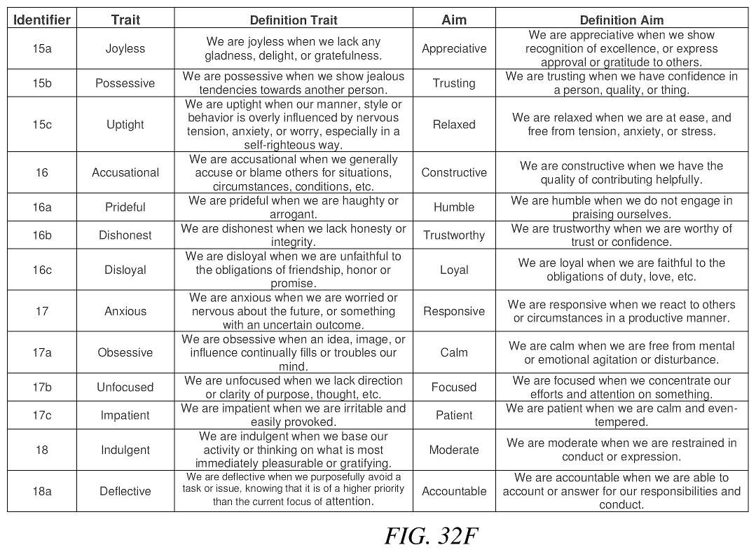

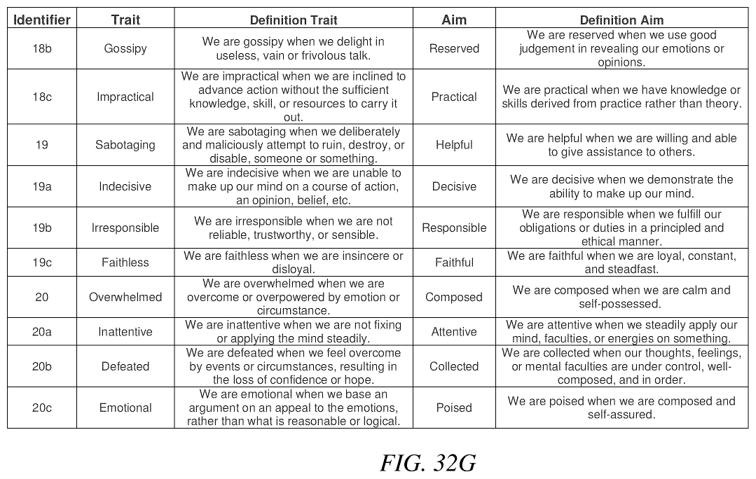

Referring to FIGS. 32A-32G, a table of traits and aims as used by a recovery trait tracker application is shown. For each row of this table, there is an identification/ID of each line for reference (e.g., 01, 01A), the name of the trait (e.g., “Undisciplined,” “Unfocused”), a definition of the trait (e.g., “We are undisciplined when we do not behave in an orderly, controlled, and effective manner”), an aim for improvement (e.g., one that desires to improve the trait of “Undisciplined” would want to be more “Disciplined”), and a definition of the aim (e.g., “We are disciplined when we are behaving in an orderly and effective manner”).

Again, it should be noted that the definitions that are used are not dictionary definitions. It is asserted that dictionary definitions will not work well as they are often to harsh for someone that is exhibiting a certain trait and wants to improve those traits.

Equivalent elements can be substituted for the ones set forth above such that they perform in substantially the same manner in substantially the same way for achieving substantially the same result.

It is believed that the system and method as described and many of its attendant advantages will be understood by the foregoing description. It is also believed that it will be apparent that various changes may be made in the form, construction, and arrangement of the components thereof without departing from the scope and spirit of the invention or without sacrificing all of its material advantages. The form herein before described being merely exemplary and explanatory embodiment thereof. It is the intention of the following claims to encompass and include such changes.

1. A method for identifying traits that need improvement and monitoring of improvements, the method comprising:
providing a set of traits;
dividing the set of traits into a plurality of primary subsets;
ranking each primary subset in the plurality of primary subsets;
removing at least one trait from each of the plurality of primary subsets;
creating a plurality of secondary subsets from the plurality of primary subsets, each secondary subset having the traits taken from each of the plurality of primary subsets;
rank ordering each secondary subset in the plurality of secondary subsets;
removing at least one trait from each of the plurality of secondary subsets;
creating a plurality of tertiary subsets from the plurality of secondary subsets, each tertiary subset having the traits taken from each of the plurality of secondary subsets;
rank ordering each tertiary subset in the plurality of tertiary subsets;
for each top-rated trait in each of the plurality of tertiary subsets, selecting two traits from either the top-rated trait or a fine-tuning trait from a list of fine-tuning traits that is associated with the top-rated trait;
for a remaining traits in each of the plurality of tertiary subsets, selecting one trait from either the remaining trait or the fine-tuning trait from the list of the fine-tuning traits that is associated with each remaining trait; and
saving the selected traits and/or the fine-tuning traits in a final subset of the traits.

2. The method of claim 1, wherein the traits are related to personal self-improvement and the set of traits comprises 30 traits, the plurality of primary subsets of the traits comprises six subsets of five traits, the plurality of secondary subsets of the traits comprises three subsets of six traits; the plurality of tertiary subsets of the traits comprises two subsets of six traits, and a number of top-rated traits is 12.

3. The method of claim 2, wherein the traits consist of undisciplined, dissatisfied, evasive, dishonest, anxious, uncharitable, unempathetic, entitled, assuming, misprioritized, insecure, impulsive, procrastinatory, lazy, uncommunicative, manipulative, judgmental, condescending, rigid, fearful, dependent, demanding, avoidant, indifferent, resentful, sabotaging, perfectionistic, unrealistic, insincere, and hypocritical.

4. The method of claim 1, wherein the traits are related to academic self-improvement and the set of traits comprises 39 traits, the plurality of primary subsets of the traits comprises nine subsets of three or five traits, the plurality of secondary subsets of the traits comprises seven subsets of three or six traits; the plurality of tertiary subsets of the traits comprises two subsets of six traits, and a number of top-rated traits is 11.

5. The method of claim 4, wherein the traits consist of disorganized, inconsistent, unproductive, undiscerning, unadaptive, dependent, unprepared, careless, lazy, emotional, undisciplined, inarticulate, uncommunicative, vulgar, apathetic, misinterpreting, unclear, imprudent, discouraged, indecisive, fearful, evasive, unmotivated, condescending, inattentive, impatient, accusational, unpolished, inefficient, hesitant, biased, indiscreet, superficial, disconcerted, unobservant, imprecise, obstinate, rude, inconsistent, and boisterous.

6. The method of claim 1, wherein the traits are related to professional self-improvement and the set of traits comprises 36 traits, the plurality of primary subsets of the traits comprises nine subsets of four traits, the plurality of secondary subsets of the traits comprises nine subsets of three traits; the plurality of tertiary subsets of the traits comprises three subsets of six traits, and a number of top-rated traits is 12.

7. The method of claim 6, wherein the traits consist of undisciplined, reticent, disorganized, impractical, misprioritized, complacent, inefficient, detached, unprepared, divisive, irresponsible, intimidated, unrealistic, unpolished, unenthusiastic, arrogant, inattentive, self-involved, laggard, uncommunicative, inaccurate, disingenuous, biased, unmindful, evasive, impetuous, indecisive, uncooperative, discouraged, showy, unassertive, indiscreet, disappointed, lazy, unadaptive, and awkward.

8. The method of claim 1, wherein the traits are related to recovering from an addiction and the set of traits comprises 20 traits, the plurality of primary subsets of the traits comprises four subsets of five traits, the plurality of secondary subsets of the traits comprises two subsets of six traits; the plurality of tertiary subsets of the traits comprises two subsets of six traits, and a number of top-rated traits is 12.

9. The method of claim 8, wherein the traits consist of impulsive, self-pitying, envious, misprioritized, rushed, controlling, unforgiving, unrealistic, dismissive, dramatic, ashamed, avoidant, dependent, entitled, rigid, accusational, anxious, indulgent, sabotaging, and overwhelmed.

10. The method of claim 1, further comprising:
periodically, for each trait in the final subset of the traits, recording progress with regard to the each trait and saving the progress in a memory.

11. The method of claim 10, further comprising:
reading the memory and presenting the progress for feedback.

12. A system for identifying traits that need improvement and for monitoring of improvements, the system comprising:
a device having a processor, a persistent storage, a non-transient memory, an input device, and a display;
a set of traits are stored in the persistent storage;
computer instructions running on the processor from the non-transient memory divides the set of traits into a plurality of primary subsets;
the computer instructions display each of the plurality of primary subsets on the display and receive inputs from the input device that provides ranks for each trait in each of the plurality of primary subsets;
the computer instructions remove at least one trait from each of the plurality of primary subsets;
the computer instructions create a plurality of secondary subsets from the plurality of primary subsets, each secondary subset has the traits taken from each of the plurality of primary subsets;
the computer instructions display each of the plurality of secondary subsets on the display and receives the inputs from the input device that provides ranks for each trait in the plurality of secondary subsets;
the computer instructions remove at least one trait from each of the plurality of secondary subsets;
the computer instructions create a plurality of tertiary subsets from the plurality of secondary subsets, each tertiary subset has the traits taken from each of the plurality of secondary subsets;
the computer instructions display each of the plurality of tertiary subsets on the display and receive the inputs from the input device that provides ranks for each trait in the plurality of tertiary subsets;
for each top-rated trait in each of the plurality of tertiary subsets, the computer instructions displays the each top-rated trait, receives the inputs selecting two traits from either the each top-rated trait or a fine-tuning trait from a list of the fine-tuning traits that is associated with the each top-rated trait, and saves selected traits in a final subset; and
for each remaining trait in each of the plurality of tertiary subsets, the computer instructions displays the each remaining trait, receives the inputs selecting one trait from either the each remaining trait or a fine-tuning trait from the list of the fine-tuning traits that is associated with the each remaining trait, and saves the selected trait in the final subset.

13. The system of claim 12, wherein the traits consist of undisciplined, dissatisfied, evasive, dishonest, anxious, uncharitable, unempathetic, entitled, assuming, misprioritized, insecure, impulsive, procrastinatory, lazy, uncommunicative, manipulative, judgmental, condescending, rigid, fearful, dependent, demanding, avoidant, indifferent, resentful, sabotaging, perfectionistic, unrealistic, insincere, and hypocritical.

14. The system of claim 12, wherein the traits consist of disorganized, inconsistent, unproductive, undiscerning, unadaptive, dependent, unprepared, careless, lazy, emotional, undisciplined, inarticulate, uncommunicative, vulgar, apathetic, misinterpreting, unclear, imprudent, discouraged, indecisive, fearful, evasive, unmotivated, condescending, inattentive, impatient, accusational, unpolished, inefficient, hesitant, biased, indiscreet, superficial, disconcerted, unobservant, imprecise, obstinate, rude, inconsistent, and boisterous.

15. The system of claim 12, wherein the traits consist of undisciplined, reticent, disorganized, impractical, misprioritized, complacent, inefficient, detached, unprepared, divisive, irresponsible, intimidated, unrealistic, unpolished, unenthusiastic, arrogant, inattentive, self-involved, laggard, uncommunicative, inaccurate, disingenuous, biased, unmindful, evasive, impetuous, indecisive, uncooperative, discouraged, showy, unassertive, indiscreet, disappointed, lazy, unadaptive, and awkward.

16. The system of claim 12, wherein the traits consist of impulsive, self-pitying, envious, misprioritized, rushed, controlling, unforgiving, unrealistic, dismissive, dramatic, ashamed, avoidant, dependent, entitled, rigid, accusational, anxious, indulgent, sabotaging, and overwhelmed.

17. The system of claim 12, further comprising:
periodically, for each trait in the final subset of the traits, the computer instructions receive progress from the input device and save the progress in a memory.

18. The system of claim 17, further comprising:
the computer instructions read the memory and present the progress for feedback.

19. Computer program product comprising: one or more non-transitory computer-readable data storage media; computing instructions stored on the one or more non-transitory computer-readable data storage media and executable by one or more processors of a device to perform:
providing a set of traits;
dividing the set of traits into a plurality of primary subsets;
ranking each primary subset in the plurality of primary subsets;
removing at least one trait from each of the plurality of primary subsets;
creating a plurality of secondary subsets from the plurality of primary subsets, each secondary subset having traits taken from each of the plurality of primary subsets;
rank ordering each secondary subset in the plurality of secondary subsets;
removing at least one trait from each of the plurality of secondary subsets;
creating a plurality of tertiary subsets from the plurality of secondary subsets, each tertiary subset having the traits taken from each of the plurality of secondary subsets;
rank ordering each tertiary subset in the plurality of tertiary subsets;
for each top-rated trait in each of the plurality of tertiary subsets, selecting two traits from either the top-rated trait or a fine-tuning trait from a list of the fine-tuning traits that is associated with each trait;
for remaining traits in each of the plurality of tertiary subset of traits, selecting one trait from either the remaining trait or the fine-tuning trait from the list of the fine-tuning traits that is associated with each trait; and
saving the selected traits and/or the fine-tuning traits in a final subset.

20. The computer program product of claim 19, wherein the set of traits consist of undisciplined, dissatisfied, evasive, dishonest, anxious, uncharitable, unempathetic, entitled, assuming, misprioritized, insecure, impulsive, procrastinatory, lazy, uncommunicative, manipulative, judgmental, condescending, rigid, fearful, dependent, demanding, avoidant, indifferent, resentful, sabotaging, perfectionistic, unrealistic, insincere, and hypocritical.
